(19) United States
(12) Patent Application Publication
(10) Pub. No.: US 2020/0157529 A1
(43) Pub. Date: May 21, 2020

(54) METHODS AND COMPOSITIONS FOR LIGAND DIRECTED ANTIBODY DESIGN

(71) Applicant: Abcam plc, Cambridge (GB)

(72) Inventors: Michael Weiner, Cambridge (GB); Margaret Kiss, Cambridge (GB); Qi Zhao, Cambridge (GB)

(21) Appl. No.: 16/635,382

(22) PCT Filed: Aug. 3, 2018
(86) PCT No.: PCT/IB2018/001000

§ 371 Date: Jan. 30, 2020

Related U.S. Application Data
(60) Provisional application No. 62/541,533, filed on Aug. 4, 2017.

(51) Int. Cl. C12N 15/10; G01N 33/68; C07K 16/40; G01N 33/573; G01N 33/563; C07K 16/00

(52) U.S. Cl. CPC C12N 15/1055; G01N 33/6803; C07K 16/40; G01N 33/573; G01N 33/563; C12N 15/1068; C07K 2317/76; C12N 15/1037; C07K 2317/622; C07K 2317/565; C07K 2317/92; C07K 2317/75; C07K 16/005

(57) ABSTRACT

The present disclosure provides, among other things, methods for generating antibodies against a target protein. In some embodiments, a library is provided comprising a plurality of tether antibodies comprising an antigen binding region and a ligand that binds to a target protein. In some embodiments, a library is provided comprising a plurality of candidate antibodies for binding to a target protein.

### RELATED APPLICATIONS

This application claims priority to U.S. Provisional Application No. 62/541,533, filed on Aug. 4, 2017, the disclosure of which is hereby incorporated by reference in its entirety.

### BACKGROUND

The development of affinity reagents that recognize and modulate membrane proteins, for example transmembrane receptors, enzymes or structural proteins by traditional animal immunization or in vitro screening methods is challenging. While select antibodies that target membrane proteins exist, the success rate of development is nonetheless poor compared with antibodies that target soluble or peripherally anchored proteins. Most of these antibodies do not modulate membrane protein function.

There remains a need for improved methods and compositions for the development of affinity reagents that recognize and modulate membrane proteins.

### SUMMARY OF THE INVENTION

Provided herein are methods to develop antibodies and antibody fragments, e.g. scFvs and IgGs, that specifically target epitopes in membrane proteins, such as, for example, GPCR, ion channel-coupled receptor, viral receptor, or enzyme-linked protein receptor, and the like. Methods and compositions disclosed herein provide a novel strategy that utilizes natural ligand affinity to generate a library of antibody variants with inherent bias toward the active site of membrane proteins, e.g. the active site of GPCRs. In some embodiments, instead of using phage libraries that display antibodies with random CDR sequences, focused antibody libraries are generated that have a natural ligand encoded within or cross-linked to one of the CDRs or the N-terminus. These methods allow for the rapid generation of antibodies (for example, both agonists and antagonists) against high value targets with poor epitope exposures including, for example, GPCRs and other integral membrane proteins.

The present disclosure provides, among other things, methods and compositions for generating antibodies against a target protein, comprising: (a) providing a tether antibody template comprising an antigen binding region and a ligand that binds to an epitope of a target protein; (b) generating a first library by randomizing one or more contact regions of the antigen binding region adjacent to a binding site between the ligand and the epitope; (c) screening the first library to identify one or more antibodies with improved binding affinity to the epitope as compared to the ligand; (d) generating a second library by randomizing a ligand carrying region of the one or more antibodies identified in step (c); (e) screening the second library to identify one or more antibodies that bind to the target protein with the same or improved affinity as compared to the ligand.

In embodiments, the epitope is a functional epitope. In embodiments, the generated antibody is an agonist or antagonist. In embodiments, the generated antibody is not an agonist or antagonist.

In some embodiments, the target protein is a membrane protein. In some embodiments, the membrane protein is a transmembrane receptor, enzyme or structural protein. In some embodiments, the transmembrane receptor is a G-protein coupled receptor (GPCR), ion channel-coupled receptor, viral receptor, or enzyme-linked protein receptor. In some embodiments, the enzyme-linked protein receptor is a receptor tyrosine kinase. In some embodiments, the functional epitope is an active site. In some embodiments, the active site is a ligand binding site. In some embodiments, the active site is a catalytic site.

In some embodiments, the antigen binding region of the tether antibody template is fused to the ligand via a peptide bond. In some embodiments, the antigen binding region of the tether antibody template is conjugated to the ligand via a covalent bond. In some embodiments, the covalent bond is a disulfide bond. In some embodiments, the tether antibody is conjugated. In embodiments, the tether antibody is conjugated by a sortase or a transglutaminase. In some embodiments, the antigen binding region of the tether antibody is an antibody fragment. In some embodiments, the antigen binding region of the tether antibody template is an scFv, Fab, Fab′, or IgG. In some embodiments, the antigen binding region of the tether antibody template is an scFv.

In some embodiments, the ligand is a peptide. In some embodiments, the ligand is a small molecule compound. In some embodiments, the ligand is fused or conjugated to a CDR of the antigen binding region. In some embodiments, the ligand is fused or conjugated to the N-terminus or C-terminus of a light chain variable region. In some embodiments, the antigen binding region is a scFv and the ligand is fused or conjugated to the C-terminus of scFv. In some embodiments, the ligand is fused or conjugated via its N-terminus or C-terminus to the antigen binding region. In some embodiments, there is a connecting loop between the antigen binding region and the ligand. In some embodiments, the connecting loop is a peptide. In some embodiments, the peptide comprises 3-50 amino acids. In some embodiments, the peptide comprises 3-21 amino acids. In some embodiments, the connecting loop is a protein.

In some embodiments, the method further comprises a step of optimizing the connecting loop. In some embodiments, the step of optimizing the connecting loop comprises screening a mini-library comprising a plurality of peptides with various lengths. In some embodiments, the connecting loop comprises an enzyme cleavage site. In some embodiments, the enzyme cleavage site is a thrombin cleavage site.

In some embodiments, prior to step (a), the method further comprises steps of designing a plurality of candidate tether antibody templates; and selecting the tether antibody template with desired binding affinity to the functional epitope. In some embodiments, the designing step comprises structural analysis of the antigen binding region and/or the ligand. In some embodiments, the plurality of candidate tether antibody templates are presented by phage display. In some embodiments, the plurality of candidate tether antibody templates are expressed as a soluble protein in the periplasm. In some embodiments, the plurality of candidate tether antibody templates are expressed as a fusion to the M13 phage coat protein gpIII.

In some embodiments, the selecting step comprises whole cell panning. In some embodiments, the selecting step comprises whole cell ELISA. In some embodiments, the desired binding affinity of the selected tether antibody template to the functional epitope has a k_{d} greater than 10 nM. In some embodiments, the one or more contact regions comprise 13-16 residues surrounding the binding site between the ligand and the functional epitope.

In some embodiments, the one or more contact regions are randomized by incorporating one or more stop codons and/or restriction enzyme cleavage sites, replacing the one or more stop codons and/or restriction enzyme sites by site directed mutagenesis resulting in a DNA template, and amplify the resulting DNA template by rolling circle amplification (RCA), thereby generating the first library. In some embodiments, the RCA is error-prone RCA. In some embodiments, the one or more contact regions are randomized without altering the ligand carrying region of the tether antibody template. In some embodiments, the first library is a phage display library. In some embodiments, the first library has a diversity of at least 10⁸, 10⁹, 10¹⁰, 10¹¹, or 10¹². In some embodiments, the step of screening the first library comprises whole cell panning. In some embodiments, the whole cell panning is emulsion based. In some embodiments, the one or more antibodies with improved binding affinity to the functional epitope are selected by competition assay using free ligands.

In some embodiments, the second library is a phage display library. In some embodiments, the second library is generated by RCA. In some embodiments, the RCA is error-prone RCA. In some embodiments, the error-prone RCA has 1-10% mutation rate. In some embodiments, the screening step of the second library comprises whole cell panning. In some embodiments, the method further comprises a step of validating the one or more ligand free antibodies identified in step (e). In some embodiments, the one or more ligand free antibodies are validated by a functional assay. In some embodiments, the step of validating the one or more ligand free antibodies identified in step (e) comprises converting scFv to IgG. In some embodiments, the method further comprises determining if the one or more ligand free antibodies are antagonistic or agonistic antibodies. In some embodiments, a functional antibody against a target protein of interest is generated. In some embodiments, a first library is generated. In some embodiments, a second library is generated.

In one aspect, a library comprising a plurality of tether antibodies comprising an antigen binding region and a ligand that binds to a a target protein, wherein the plurality of tether antibodies are derived from a tether antibody template and comprise randomized one or more contact regions adjacent to a binding site of the ligand and an epitope of the target protein. In embodiments, the epitope is a functional epitope. In some embodiments, the plurality of tether antibodies comprises an unaltered ligand carrying region. In some embodiments, the antigen binding region is fused to the ligand via a peptide bond. In some embodiments, antigen binding region is conjugated to the ligand via a covalent bond. In some embodiments, the covalent bond is a disulfide bond. In some embodiments, the antigen binding region is an antibody fragment. In some embodiments, the antigen binding region is a scFv, Fab, Fab′, or IgG. In some embodiments, the antigen binding region is a scFv.

In some embodiments, the ligand is a peptide. In some embodiments, the ligand is a small molecule compound. In some embodiments, the ligand is a polymer, DNA, RNA or sugar. In some embodiments, the ligand is fused or conjugated to a CDR of the antigen binding region. In some embodiments, the ligand is fused or conjugated to the N-terminus or C-terminus of a light chain variable region. In some embodiments, the antigen binding region is a scFv and the ligand is fused or conjugated to the C-terminus of scFv. In some embodiments, the ligand is fused or conjugated via its N-terminus or C-terminus to the antigen binding region.

In some embodiments, there is a connecting loop between the antigen binding region and the ligand. In some embodiments, the connecting loop is a peptide. In some embodiments, the peptide comprises 3-50 amino acids. In some embodiments, the peptide comprises 3-21 amino acids. In some embodiments, the connecting loop comprises an enzyme cleavage site. In some embodiments, the enzyme cleavage site is a thrombin cleavage site. In some embodiments, the library is a phage display library. In some embodiments, the plurality of tether antibodies are expressed as a soluble protein in the periplasm. In some embodiments, the plurality of tether antibodies are expressed as a fusion to the M13 phage coat protein gpIII. In some embodiments, the library has a diversity of at least 10⁸, 10⁹, 10¹⁰, 10¹¹, or 10¹².

In one aspect, a library is provided comprising a plurality of candidate antibodies for binding to a a target protein, wherein the plurality of the candidate antibodies are derived from a parent antibody comprising one or more contact regions adjacent to a binding site between a ligand and an epitope of the target protein and a ligand carrying region that contacts the epitope and competes with the ligand, wherein the plurality of candidate antibodies comprise randomized ligand carrying region. In embodiments, the epitope is a functional epitope.

In some embodiments, the plurality of candidate antibodies comprise substantially identical one or more contact regions. In some embodiments, the library is a phage display library. In some embodiments, the plurality of candidate antibodies are expressed as a soluble protein in the periplasm. In some embodiments, the plurality of tether antibodies are expressed as a fusion to the M13 phage coat protein gpIII. In some embodiments, the library has a diversity of at least 10⁸, 10⁹, 10¹⁰, 10¹¹, or 10¹².

In one aspect, a method is provided for generating binders against a target protein, comprising (a) providing a tether antibody template comprising an antigen binding region and a ligand that binds to an epitope of a target protein; (b) generating a first library by randomizing one or more contact regions of the antigen binding region adjacent to a binding site between the ligand and the epitope; (c) screening the first library to identify one or more binders with improved binding affinity to the epitope as compared to the ligand; (d) generating a second library by randomizing a ligand carrying region of the one or more binders identified in step (c); (e) screening the second library to identify one or more binders that bind to the target protein with the same or improved affinity as compared to the ligand. In embodiments, the ligand is a peptidomimetic or aptamer.

As used in this application, the terms “about” and “approximately” are used as equivalents. Any citations to publications, patents, or patent applications herein are incorporated by reference in their entirety. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWING

FIG. 1, panels a-d, is a series of schematics that indicate a workflow of ligand-directed antibody design. FIG. 1, panel a, is a schematic that shows an exemplary ligand-receptor pair. Upon identifying the ligand-receptor pair, a template is designed and validated (FIG. 1, panel b). This step includes the generation of the initial tether. Following initial tether generation, there is a first round screening process that occurs, wherein a first library is generated and screened by randomizing possible contact regions of the tether to the receptor (FIG. 1, panel c). This is followed by a second library generation and screening via mutation within ligand-carrying regions (FIG. 1, panel d).

FIG. 2, panels a-d, is a series of schematics that show a structural-based template design and choice of randomizing regions for first round screening. FIG. 2, panel a, is a schematic of a model receptor AT1R (PDBID: 4YAY, TM and cellular/ecto domains as ribbons, and surface loops and the natural ligand is modelled in the binding pocket: angiotensin II). FIG. 2, panel b, is a schematic of the deep substrate binding pocket (cross-section view of AT1R Van de Waals surface view). FIG. 2, panel c, is a schematic that shows the design of the ligand connecting loop, three possible attaching points and possible contacting regions on CDR on scFv as ribbons. FIG. 2, top half of panel d, shows the over view of the possible interacting regions in the scFv. FIG. 2, bottom half of panel d, shows the cross-section view along the direction of transmembrane (TM) helices, the proposed interacting regions as ribbons also overlap well with the AT1R surface exposed regions displayed as surfaces.

FIG. 3 is a schematic (panel a) and a bar graph (panel b) that depicts data obtained from three formats of ligand-phage/scFv template binding to target. FIG. 3, panel a, shows the design of three formats of ligand-phage/scFv. FIG. 3, panel b, shows a bar graph that depicts data obtained from whole cell ELISA data using three formats and two thrombin treated versions of the formats. The negative control used was anti-M13 HRP only. The fractional occupancy of binding sites (“FOB”) was calculated by dividing the ELISA 425 nM illuminances signal of phage applied to AT1R(+) cells over AT1R(−) cells (5×10⁵ transient AT1R expression HEK293T cells per well were used in this assay).

FIG. 4, panel a-b, is a series of schematics and graphs that depict a rolling circle amplification (RCA) library generation pipeline and a comparison with traditional Kunkel mutagenesis-based library generation. FIG. 4, panel a, is a series of schematics that depicts RCA library generation and Kunkel mutagenesis-based library generation. The library generated by traditional methods will be selectively amplified by approximately 100 fold using Rolling circle amplification (RCA), linearization and re circularization. FIG. 4, panel b, is a series of graphs that depict DNA concentration, recombinant rate, colonies per transformation, and diversity per transformation of RCA library generation in comparison to traditional library generation. The data indicate that when transformed into TG1 cells, the new library displayed superior efficiency over the starting library in the aspect of total colonies per transformation and realized diversity, taking into account the multiplicity of transformation. (*) transformed using TG1 cells.

FIG. 5, panels a-d, is a series of schematics and graphs that depict methods to increase diversity and affinity maturation. FIG. 5, panel a, depicts phage micro-emulsion technology using whole cells for an exemplary antibody against a cell surface receptor. Within a microdroplet, phage-transduced E. coli attach to an antigen-coated bead or cell (SF9, mammalian, Rhodobacter, Arabidopsis, etc.) expressing a membrane spanning protein. After overnight incubation, the emulsion is broken, the beads or cells are washed, and a FITC-labelled anti-M13 Ab is added to detect bound phage. The beads or cells are then sorted by FACS (FIG. 5, panel b). The beads or cells are validated by using whole cell ELISA (FIG. 5, panel c) and a functional competition assay (FIG. 5, panel c, right graph) (NC1: irrelevant scFv, PC1, commercial monoclonal antibody, NC2, no scFv). FIG. 5, panel d, depicts a schematic of a method to enhance enrichment for whole cell panning via induced hexamerization. Hexamerizing protein (TH7) (PDB entry ID: 2m3x) can be genetically linked to cytoplasmic or extracellular domain of GPCR to enhance avidity. Experimentally (right panel), TH7 was genetically linked to the extracellular domain of OmpA. An antigenic peptide (FLAG peptide) was genetically linked to the C terminal of each TH7 subunit to serve as a multivalent antigen display platform (OmpA-TH7-linker-FLAG) on the E. coli outer membrane. Using E. coli cells carrying this displaying system in whole cell panning showed superior enrichment within one round of cell panning. In comparison, traditional monovalent display system (OmpA-linker-FLAG) did not show observable enrichment.

FIG. 6 depicts a series of schematics that show sortase chemistry, site-specific conjugation. FIG. 6 shows a schematic of site-specific C-terminal and internal loop labelling of a protein using sortase-mediated reactions.

FIG. 7, panels a-b, depicts a schematic of AXM affinity maturation mutagenesis procedure (panel a), and exemplary maturation outcome (panel b). The results show that μM to nM of Kd change was achieved through the use of AXM affinity maturation mutagenesis.

FIG. 8 is a series of FACS graphs of Neurotensin Receptor Type 1 (NTSR1) ligand libraries that show increased binding to NTSR1 cells following multiple rounds of screening. R1=round 1. R2=round 2. R3=round 3. FIG. 8 provides monitoring of the improvement of panning with rounds using polyclonal phage FACS FITC assay for multiple panning on NTSR1.

FIG. 9 is a series of FACS graphs that show two exemplary, strong anti-NTSR1 phage hits.

FIG. 10 is a series of FACS graphs that show data relating to five-selected weak phage hits from an NTSR1 library screen.

FIG. 11 is a bar graph and a series of FACS graphs that show a difference in phage titers between weak and strong hits from an NTSR1 library screen.

FIG. 12 is a series of FACS graphs that show data obtained from four libraries with coupled NTSR2 ligand.

FIG. 13, panels A-D, are a series of graphs that show the validation of isolated NTSR1 and NTSR2 binders. FIG. 13, panels A and B show the characterization of the isolated NSTR1 and NTSR2 binders by flow cytometry. FIG. 13C and FIG. 13D show a series of graphs that indicate that the isolated NTSR1 and NTSR2 binders are functional as assessed by a calcium assay agonist/antagonist assay.

FIG. 14, panels A and B is a series of flow cytometry graphs that indicate isolated NTSR1 binders that have undergone affinity maturation bind with high affinity as monovalent phage on NTSR1 expressing cells (panels A and B). The flow cytometry graphs also indicate that the affinity matured NTSR1 binders bind tighter than those that have not been affinity matured.

FIG. 15, panels A and B, are a series of flow cytometry graphs that show improved specificity of NTSR1 (panel A) and NTSR 2 (panel B) following affinity maturation.

FIG. 16, panels A-D, are a series of flow cytometry graphs that show binding specificity of isolated CXCR4 binders. The data presented in FIG. 16, panels C, shows CXCR4 polyclonal phage FACS analysis indicating phage binding after bulk panning and round 1 of whole cell panning (left graph, FIG. 16, panel C) and an increase in phage binding after the second round of whole cell panning (right graph, FIG. 16, panel C). FIG. 16, panel D, depicts a flow cytometry graph of a CXCR4 phage that was comprised of a mixed population of clones.

FIG. 17 is a series of graphs indicating that the isolated CXCR4 binders exhibit strong antagonist properties.

### DEFINITIONS

In order for the present invention to be more readily understood, certain terms are first defined below. Additional definitions for the following terms and other terms are set forth throughout the specification.

Affinity regent: As used herein, the term “affinity reagent” is any molecule that specifically binds to a target molecule, for example, to identify, track, capture or influence the activity of the target molecule. The affinity reagent identified or recovered by the methods described herein are “genetically encoded,” for example an antibody, peptide or nucleic acid, and are thus capable of being sequenced. The terms “protein,” “polypeptide,” and “peptide” are used interchangeably herein to refer to two or more amino acids linked together.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Antibody: As used herein, the term “antibody” refers to immunoglobulin molecules and immunologically active portions of immunoglobulin (Ig) molecules, i.e., molecules that contain an antigen binding site that binds (immunoreacts with) an antigen. By “binds” or “immunoreacts with” is meant that the antibody reacts with one or more antigenic determinants of the desired. Antibodies include, antibody fragments. Antibodies also include, but are not limited to, polyclonal, monoclonal, chimeric dAb (domain antibody), single chain, F_{ab}, F_{ab}′, F_{(ab′)2} fragments, scFvs, and F_{ab} expression libraries. An antibody may be a whole antibody, or immunoglobulin, or an antibody fragment.

Antigen binding site: As used herein, the term “antigen-binding site,” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Binder: As used herein, a “binder” refers to any compound, naturally occurring or non-naturally occurring that is capable of binding a target. In embodiments, a “binder” is a small molecule, an antibody or other chemical compound or moiety.

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a peptide is biologically active, a portion of that peptide that shares at least one biological activity of the peptide is typically referred to as a “biologically active” portion.

Epitope: As used herein, the term “epitope”” includes any protein determinant capable of specific binding to an immunoglobulin, or fragment. Epitopic determinants usually consist of chemically active surface groupings of molecules such as amino acids or sugar side chains and usually have specific three dimensional structural characteristics, as well as specific charge characteristics. For example, antibodies may be raised against N-terminal or C-terminal peptides of a polypeptide.

Functional epitope: As used herein, the term “functional epitope” means the residues within the epitope that make energetic contributions to the binding interaction and/or involved in any physiological or biochemical function of the protein.

Functional equivalent or derivative: As used herein, the term “functional equivalent” or “functional derivative” denotes, in the context of a functional derivative of an amino acid sequence, a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

GPCRs: As used herein, GPCRs (G-Protein Coupled Receptors) are a group of integral membrane proteins with 7 transmembrane (7TM) helices.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, about 95%, about 98%, about 99%, substantially 100%, or 100% of the other components with which they were initially associated. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components. As used herein, the term “isolated cell” refers to a cell not contained in a multi-cellular organism.

Immunological Binding: The term “immunological binding” refers to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein smaller Kd represents a greater affinity. Immunological binding properties of selected polypeptides can be quantified using methods well known in the art.

Molecular Display System: As used herein, the term “molecular display system” is any system capable of presenting a library of potential affinity reagents to screen for potential binders to a target molecule or ligand. Examples of molecular display systems include phage display, bacterial display, yeast display, ribosome display and mRNA display. In some embodiments, phage display is used.

Polypeptide: The term “polypeptide” as used herein refers a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified.

Peptidomimetic: The term “peptidomimetic” refers to any compound that mimics a peptide. This can be a first peptide that mimics the binding or functionality of an unrelated ligand peptide, but with a substantially different sequence. The peptidomimetic may be any engineered or naturally occurring compound. In embodiments, the peptidomimetic is a peptidomimetic macrocyle which is a compound comprising a plurality of amino acid residues joined by a plurality of peptide bonds and at least one macrocycle-forming linker which forms a macrocycle between a first naturally occurring or non-naturally occurring amino acid residue (or analog) and a second naturally-occuring or non-naturally-occuring amino acid residue (or analog) within the same molecule.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

scFv: A single chain Fv (“scFv”) polypeptide molecule is a covalently linked VH::VL heterodimer, which can be expressed from a gene fusion including VH- and VL-encoding genes linked by a peptide-encoding linker. (See Huston et al. (1988) Proc Nat Acad Sci USA 85(16):5879-5883). A number of methods have been described to discern chemical structures for converting the naturally aggregated, but chemically separated, light and heavy polypeptide chains from an antibody V region into an scFv molecule, which will fold into a three dimensional structure substantially similar to the structure of an antigen-binding site. See, e.g., U.S. Pat. Nos. 5,091,513; 5,132,405; and 4,946,778.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutic Protein Target: As used herein, the term “therapeutic protein target” or “biological target” means anything within a living organism (e.g. a cell, protein, small molecule, RNA, DNA and the like) to which some other entity is directed and/or binds, wherein the binding changes the living organism's physiology.

### DETAILED DESCRIPTION OF CERTAIN EMBODIMENTS

Various aspects of the invention are described in detail in the following sections. The use of sections is not meant to limit the invention. Each section can apply to any aspect of the invention. In this application, the use of “or” means “and/or” unless stated otherwise.

The present disclosure provides, among other things, methods and compositions for generating binders, including antibodies, against a target protein. The target protein can be any protein of interest. In embodiments, the target protein is a GPCR, a cell surface protein, an isolated protein, or a therapeutic protein target. Examples of therapeutic targets (e.g. targets that regulate the physiology of a cell and/or an organism) are known by those of skill in the art. In embodiments, the methods provided herein allow for the discovery of binders, including antibodies that interact with a target peptide's active site, or regions that are adjacent to the active site. In embodiments, the methods provided herein allow for the discovery of binders that interact with regions of the target protein that are distant from the active site of the target protein.

The methods provided herein therefore allow for the discovery of binders that can bind to any epitope of the target protein or peptide. In embodiments, the binders, once bound to the target protein or peptide, do not serve as an agonist or an antagonist. In embodiments, the binders once bound to the target protein or peptide, serve as an agonist or an antagonist. In embodiments, the methods provided herein allow for the isolation of binders that can serve as allosteric or competitive inhibitors of a protein target. For example, the methods herein allow for the isolation of binders that can be used to modify the activity of a protein target. Such modification of the activity of a protein target includes the upregulation, downregulation or ablation of the activity associated with a protein target.

The methods provided herein can also be utilized in combination with peptidomimetics or aptamers to find binders with high binding affinity. For example, a peptidomimetic is first discovered and isolated through means known in the art. The isolated peptidomimetic or aptamer can, for example, bind to a receptor or other peptide or protein. In embodiments, the isolated peptidomimetic or aptamer is a functional inhibitor or activator. In embodiments, the isolated peptidomimetic or aptamer once bound to its target does not inhibit or activate any function in the bound target. In embodiments, the isolated peptidomimetic or aptamer is incorporated into the CDR of the antibody library, followed by screening the antibodies in the library that are able to bind with high affinity. In embodiments, the peptidomimetic or aptamer is enzymatically ligated into the antibodies of the library. As explained more fully below, various methods of enzymatic ligation can be used, for example through the use of sortases (recognizing “LPXTG”) or transglutaminases (recognizing a glutamine harbored by up to 6 specific amino acids on both sides).

In embodiments, the methods provided herein allow for the generation of antibodies that target a functional epitope of a target protein. In this way, such targeting of a functional epitope of a target protein allows the antibody to modulate the target protein's function. For example, the ability to target a functional epitope allows the antibody to agonize or antagonize the target protein's function. In some embodiments, the methods provided herein use ligand-conjugated antibody libraries for the development of antibodies that can modulate a target protein's function.

### Target Proteins

Any protein can be a target protein. In some embodiments, an integral membrane protein is the target protein. Integral membrane proteins contain one or more regions which completely span the cell membrane. Often these molecules constitute important cell surface recognition or signaling molecules. Examples of integral membrane proteins include G protein-coupled receptors, which classically have 7 transmembrane spanning regions, and ion channels and gates, whose pore-forming subunits typically have multiple transmembrane domains. Specific, non-limiting, examples of integral membrane proteins include, for example, receptor tyrosine kinases, insulin, select cell adhesion molecules (CAMs) including integrins, cadherins, NCAMs, and selectins, glycophorin, rhodopsin, CD36, GPR30, glucose permease, gap junction proteins, and seipin.

In some embodiments, the target protein is an integral membrane protein, such as, for example, G-protein coupled receptors (GPCRs), ion channel-coupled receptors, viral receptors, or enzyme-linked protein receptors, and the like. Membrane-proteins such as GPCRs are involved in the regulation of many biological functions. GPCRs regulate sensory perception, cell-growth and hormonal responses. They are targets for over 40% of current prescription drugs, and the market for these drugs is over $100 Billion worldwide (2014 data). The ability to agonize or antagonize GPCR function is a central issue for both basic research and pharmaceutical applications. A variety of agents, i.e. chemicals or biologics, have been explored to lock this integral membrane protein in its active or inactive conformation. Antibodies and single-chain antibody fragments (scFv) emerged as promising tools owing to their biocompatibility, superior specificity and robustness of development. Functional antibodies, those that are capable of not only binding to the receptor but also of modulating its function, are of high pharmaceutical value. Herein is disclosed a method to develop scFvs and IgGs that specifically target the functional epitope of a target protein.

In some embodiments, the target protein is a GPCR. The GPCR can be any GPCR. As non-limiting examples, the GPCR can be 5-Hydroxytryptamine receptors, Acetylcholine receptors (muscarinic), Adenosine receptors, Adhesion Class GPCRs, Adrenoceptors, Angiotensin receptors, Apelin receptor, Bile acid receptor, Bombesin receptors, Bradykinin receptors, Calcitonin receptors, Calcium-sensing receptor, Cannabinoid receptors, Chemerin receptor, Chemokine receptors, Cholecystokinin receptors, Class Frizzled GPCRs, Complement peptide receptors, Corticotropin-releasing factor receptors, Dopamine receptors, Endothelin receptors, G protein-coupled estrogen receptor, Formylpeptide receptors, Free fatty acid receptors, GABAB receptors, Galanin receptors, Ghrelin receptor, Glucagon receptor family, Glycoprotein hormone receptors, Gonadotrophin-releasing hormone receptors, GPR18, GPR55 and GPR119, Histamine receptors, Hydroxycarboxylic acid receptors, Kisspeptin receptor, Leukotriene receptors, Lysophospholipid (LPA) receptors, Lysophospholipid (S1P) receptors, Melanin-concentrating hormone receptors, Melanocortin receptors, Melatonin receptors, Metabotropic glutamate receptors, Motilin receptor, Neuromedin U receptors, Neuropeptide FF/neuropeptide AF receptors, Neuropeptide S receptor, Neuropeptide W/neuropeptide B receptors, Neuropeptide Y receptors, Neurotensin receptors, Opioid receptors, Orexin receptors, Oxoglutarate receptor, P2Y receptors, Parathyroid hormone receptors, Platelet-activating factor receptor, Prokineticin receptors, Prolactin-releasing peptide receptor, Prostanoid receptors, Proteinase-activated receptors, QRFP receptor, Relaxin family peptide receptors, Somatostatin receptors, Succinate receptor, Tachykinin receptors Thyrotropin-releasing hormone receptors, Trace amine receptor, Urotensin receptor, Vasopressin and oxytocin receptors, VIP and/or PACAP receptors.

In some embodiments, the target protein is selected from lipases, proteases, kinases, sortases, and/or Cas9.

In certain embodiments, the target protein is a therapeutic protein target or a biological target. Therapeutic protein targets or biological targets that can be manipulated to achieve a certain physiological effect in an organism are known in the art. Various categories of therapeutic proteins are known in the art, for example, anticoagulants, blood factors, bone morphogenetic proteins, engineered protein scaffolds, enzymes, growth factors, hormones, interferons, interleukins, and thrombolytics.

### Ligand-Directed Antibodies

Recombinant antibodies (rAb), like single chain variable fragments (scFv), have many attractive attributes compared to polyclonal antisera and monoclonal antibodies derived from hybridomas. They are renewable through overexpression in the appropriate heterologous host, they are easily stored and transferred as DNA, and they can be genetically engineered as fusions to various enzymes, fluorescent proteins, and epitope tags. However, in vitro selection methods, such as phage display, yeast display, and ribosome display have thus far been inefficient at meeting the need for customized antibodies that target membrane proteins. Several methods have been used to create antibody diversity in vitro. These include cloning cDNA of the immune regions (a native approach) of either immunized or non-immunized vertebrate cells (to create a naïve library), total synthesis of antibody CDR gene fragments with mixed-nucleotide synthesis, and a semi-synthetic approach whereby a framework gene is synthesized, and the diversity is generated by cloning a multitude of CDRs. Disadvantages to these library types include variable biophysical properties and expression levels using native libraries with heterogeneous frameworks, and stop codons in mixed-nucleotide sequences in synthetic and semi-synthetic approaches. However, the total potential diversity with these libraries is still substantially higher (>10²³) than the diversity that can be sampled (typically 10₁₁-10¹² with phage libraries), and not all amino acids at a given CDR position will yield a folded antibody.

Described herein is a method to generate ligand-directed antibodies. Ligand-directed antibodies make use of ligand-target interactions to specifically target the functional epitope of a protein of interest. In some embodiments, the functional epitope is an active site, ligand binding site or catalytic site. The generation of a directed-ligand antibody includes: 1) providing a tether antibody template comprising an antigen binding region and a ligand that binds to a functional epitope of a target protein; 2) generating a first library by randomizing one or more contact regions of the antigen binding region adjacent to a binding site between the ligand and the functional epitope; 3) screening the first library to identify one or more antibodies with improved binding affinity to the functional epitope in comparison to the ligand; 4) generating a second library by randomizing a ligand carrying region of the one or more antibodies identified in the previous step; and 5) screening the second library to identify one or more antibodies that bind to the functional epitope with the same or improved affinity in comparison to the ligand.

### Ligand Receptor Pair Identification and Fusion of Tether Antibody Template to Ligand

In some embodiments, the initial step in the generation of ligand-directed antibodies is the identification of a ligand-receptor pair. The ligand of interest can be any compound. For example, the ligand of interest can be a peptide, or a small molecule compound. In some embodiments, the ligand can be a polymer, DNA, RNA or a sugar. In embodiments, the ligand is a peptidomimetic or aptamer. Methods for identifying a ligand-receptor pair include structural analysis of the antigen binding region in the peptide or protein of interest and/or in the ligand of interest. Upon identification of a suitable ligand-receptor pair, the ligand is fused to a tether antibody template. The ligand can be fused or conjugated to a CDR, or to the N-terminus or C-terminus of a light chain variable region. In embodiments, the fusion is performed enzymatically, with, for example, sortases or transglutaminases. In some embodiments, the antigen binding region is a scFv and the ligand is fused or conjugated to the C-terminus of scFv. In some embodiments, the ligand is fused or conjugated via its N-terminus or C-terminus to the antigen binding region.

There are numerous manners to fuse or conjugate the tether antibody template to the ligand of interest. The tether antibody template can be any antibody or antibody fragment.

Any manner known in the art can be used to generate a fusion or conjugation between the tether antibody template and the ligand of interest. In some embodiments, the antigen binding region of the tether antibody template is fused or conjugated to the ligand through a peptide bond, covalent bond, disulfide bond, or ester bond. In some embodiments, sortases (recognizing “LPXTG”) or transglutaminases (recognizing a glutamine harbored by up to 6 specific amino acids on both sides) are used to fuse the tether antibody template to the ligand of interest. Sortases allow for site-specific fusion of the tether antibody template to the ligand. The use of sortases allows for similar precision to genetic fusion methods and provides access to protein derivative structures that are unattainable genetically. Naturally occurring sortases are selective for specific C-terminal and N-terminal recognition motif amino acid sequence LPXTG, where X represents any amino acid. The T and the G in the substrate can be connected using a peptide bond or an ester linkage. In some embodiments, a sortase recognition sequence is engineered to allow for fusion of the tether antibody to the ligand of interest.

### First Library Generation

In methods described herein, a first antibody library is generated. In some embodiments, the first antibody library is a phage library. The phage used in the phage library can be any phage. In some embodiments, the phage used is M13, fd filamentous phage, T4, T7 or λ, phage. In some embodiments, the phage used in the phage library is M13 phage. In some embodiments, the tether antibody templates are expressed on a selected phage coat protein. Suitable phage coat proteins are known in the art. In some embodiments, the phage coat protein is gpIII.

Generation of the first antibody library can be made through any method known in the art. In some embodiments, a first library is generated by randomizing one or more contact regions of the antigen binding region adjacent to a binding site between the ligand and the functional epitope. In this manner, the one or more contact regions are randomized without altering the ligand carrying region of the tether antibody template. In some embodiments, the contact regions can be one or more complementarity determining regions (CDRs) that are selected for mutagenesis. In some embodiments, stop codons and/or restriction enzyme cleavage sites are incorporated into the selected CDRs. The stop codons and restriction enzyme cleavage sites are replaced through site-directed mutagenesis. Any kind of site-directed mutagenesis known in the art can be used. In some embodiments, Kunkel-based mutagenesis is used to replace incorporated stop codons and/or restriction enzyme recognition sites with tri-oligonucleotides that encode naturally distributed sets of residues at selected CDR positions. The resulting DNA template is then amplified.

Any kind of library amplification methods known in the art can be used for library amplification. In some embodiments, a sequence of interest may be amplified using a pair of oligonucleotides, of which one oligonucleotide is a protected oligonucleotide and the other is a non-protected oligonucleotide. The sequence of interest may be amplified using such an oligonucleotide pair by an amplification reaction such as PCR, error-prone PCR, isothermal amplification, or rolling circle amplification. In some embodiments, rolling circle amplification (RCA) is used. In some embodiments, error-prone rolling circle amplification is used. RCA can amplify the library by about between 50- and 150-fold (e.g. 50, 55, 60, 65, 70, 75, 80, 85, 90, 95, 100, 105, 110, 115, 120, 125, 130, 135, 140, 145, 150, and any values in between). In some embodiments, RCA can amplify the library by about 100-fold. In some embodiments, the RCA amplified library is linearized and re-circularized.

The ligand-antibody library may be introduced into any suitable cell known in the art. The cell may be an archaeal cell, prokaryotic cell, bacterial cell, fungal cell, or eukaryotic cell. The cell may be a yeast cell, plant cell, or animal cell. In some instances, the cell may be an E. coli cell or S. cerevisiae cell. The cell strain can be any electro- or chemical competent cell. In some embodiments, the library can be transformed into DH5α, JM109, C600, HB101, or TG1. In some embodiments, the library is transformed into TG1 cells. In some embodiments, the plurality of tether antibodies is expressed as a soluble protein in the periplasm.

In some embodiments, the first library has a diversity of about between 10⁷ and 10¹⁴ (i.e. 10⁷, 10⁸, 10⁹, 10¹⁰, 10¹¹, 10¹², 10¹³, and 10¹⁴) unique ligand-antibodies. In some embodiments, the first library has a diversity of at least 10⁸ and 10¹² (i.e. 10⁸, 10⁹, 10¹⁰, 10¹¹, 10¹²).

### Screening and Validating the First Library

The first library is screened to identify one or more antibodies with improved binding affinity to the functional epitope. Any method known in the art can be used to screen the first library for binding to the functional epitope. In some embodiments, an emulsion whole cell based library screening method is used. Whole cell screening methods (e.g. whole cell panning) are described in US patent application publication number 201503322150, the contents of which are hereby incorporated by reference in its entirety. Whole cell screening methods include, for example, the creation of an emulsion in which E. coli that have been transduced with the ligand-antibody phage library are incubated with cells or beads that display the antigen of interest. During an overnight incubation process, ligand-antibody displaying phages are secreted from the E. coli and attach to the antigen presenting cells or beads. Subsequent processing includes the addition of labeled antibodies that attach to the phage, and subsequent FACS sorting to isolate the ligand-antibody displaying phage that have bound to the antigens displayed on the whole cell or beads. In some embodiments, the library is processed for multiple rounds of whole cell screening. In some embodiments, whole cell screening is performed between about 3 to 8 times (i.e. 3, 4, 5, 6, 7, 8). In some embodiments, whole cell screening is performed about 3 times. In some embodiments, multiple rounds of whole cell screening results in the isolation of more specific epitope binding antibodies.

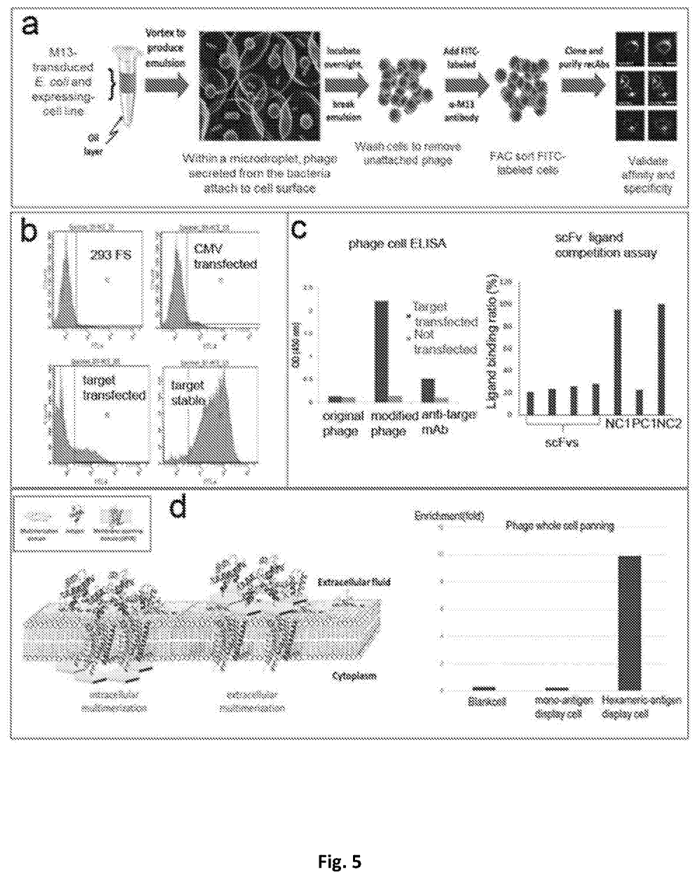

In some embodiments, the isolated ligand-antibodies are further validated by use of ELISA and functional competition assays. The competition assay, can include, for example, a competition assay using free ligands. These further validations are intended to ascertain that the binding of the antibody to the epitope is improved in comparison to the binding of ligand alone. In further embodiments, methods to enhance enrichment for whole cell panning can be used. For example, in some embodiments, enrichment for whole cell panning is accomplished via induced hexamerization. Hexamerization is performed by genetically linking hexamerizing protein (TH7) to cytoplasmic or extracellular domain of a membrane protein to enhance avidity. The creation of an OmpA-TH7-linker-FLAG on the cells' outer membrane enriches whole cell panning. See FIG. 5, panel d.

### Second Library Generation and Screening

Following the isolation of screened and validated binders from the first library, a second library is generated. In some embodiments, the second library is a phage library. The purpose of the second library is to eliminate, reduce and/or phase out the affinity contributed by the ligand in the isolated ligand-antibodies. To this end, mutations will be introduced into the ligand carrying region, which was not mutated in the first library.

In some embodiments, two randomization strategies are used, and the end products are combined to produce the second round library.

In some embodiments, the first randomization strategy introduces about a 1 to 10% mutation rate (i.e. 1.0, 1.5. 2.0, 2.5. 3.0, 3.5, 4.0, 4.5, 5.0, 5.5, 6.0, 6.5, 7.0, 7.5, 8.0, 8.5, 9.0, 9.5, 10, or any values in between) to the ligand and its flanking region using any method known in the art. In some embodiments, the first randomization strategy introduces a 2-5% (i.e. 2.0, 2.5, 3.0, 3.5, 4.0, 4.5, 5.0, or any values in between) mutation rate to the ligand and its flanking region using any method known in the art. In some embodiments, the mutations are introduced by error-prone PCR.

In some embodiments, the second randomization strategy introduces segmental randomization. In some embodiments, segmental randomization uses an NNK randomization scanning window of 9 nucleotides (or 3 amino acids) that is applied on the ligand and at −4aa and +4aa of the flanking regions.

Any kind of library amplification methods known in the art can be used for amplification of the second library. In some embodiments, a sequence of interest may be amplified using a pair of oligonucleotides, of which one oligonucleotide is a protected oligonucleotide and the other is a non-protected oligonucleotide. The sequence of interest may be amplified using such an oligonucleotide pair by an amplification reaction such as PCR, error-prone PCR, isothermal amplification, or rolling circle amplification. In some embodiments, rolling circle amplification (RCA) is used. In some embodiments, error-prone rolling circle amplification is used. RCA can amplify the library by about between 50- and 150-fold (e.g. 50, 55, 60, 65, 70, 75, 80, 85, 90, 95, 100, 105, 110, 115, 120, 125, 130, 135, 140, 145, 150, and any values in between). In some embodiments, RCA can amplify the library by about 100-fold. In some embodiments, the RCA amplified library is linearized and re-circularized.

The ligand-antibody library can be introduced into any suitable cell known in the art. The cell may be an archaeal cell, prokaryotic cell, bacterial cell, fungal cell, or eukaryotic cell. The cell may be a yeast cell, plant cell, or animal cell. In some instances, the cell may be an E. coli cell or S. cerevisiae cell. The cell strain can be any electro- or chemical competent cell. In some embodiments, the library can be transformed into DH5α, JM109, C600, HB101, or TG1. In some embodiments, the library is transformed into TG1 cells.

In some embodiments, the second library has a diversity of about between 10⁷ and 10¹⁴ (i.e. 10⁷, 10⁸, 10⁹, 10¹⁰, 10¹¹, 10¹², 10¹³, and 10¹⁴) unique ligand-antibodies. In some embodiments, the first library has a diversity of at least 10⁸ and 10¹² (i.e. 10⁸, 10⁹, 10¹⁰, 10¹¹, 10¹²).

In some embodiments, the second library is screened by the whole cell based library screening method. In some embodiments, the library is processed for multiple rounds of whole cell screening. In some embodiments, whole cell screening is performed between about 3 to 8 times (i.e. 3, 4, 5, 6, 7, 8). In some embodiments, whole cell screening is performed 3 times. In some embodiments, multiple rounds of whole cell screening results in the isolation of more specific epitope binding antibodies.

The second library binders are further validated by use of ELISA and functional competition assays. In some embodiments, the isolated clones that have an ELISA signal greater than 2-fold over background will be expressed in E. coli and purified by metal chromatography. In some embodiments, a further functional validation is performed on the isolated second library binders. Any method known in the art can used to validate the second library binders.

### Connecting Loop

In some embodiments, the methods herein use an antibody tethered to a ligand by a connecting loop (also referred to herein as a “tether” or “tether loop”). The connecting loop is found between the antigen binding region of the antibody and the ligand. The connecting loop can be a peptide, polypeptide or protein. In some embodiments, the connecting loop is a peptide.

In some embodiments, the length of the connecting loop is between about 3 to 50 (i.e. 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) amino acids. In some embodiments, the length of the connecting loop is between about 3 to 21 (i.e. 3, 4, 5, 6, 7, 8, 9, 10, 11, 12, 13, 14, 15, 16, 17, 18, 19, 20, or 21) amino acids.

In some embodiments, the connecting loop is optimized to enhance binding of the antibody and/or ligand. In some embodiments, the length of the connecting loop is optimized by screening a mini-library that includes a plurality of connecting loop peptides having various lengths. The mini-library is constructed from ssDNA from the template that had the best affinity in cell ELISA validation. In some embodiments, an oligo set that carries a random length central region of about between 3 and 80 nucleotides (i.e. 3, 4, 5, 6, 7, 8, 9, 10, 15, 20, 25, 30, 35, 40, 45, 50, 55, 60, 65, 66, 67, 68, 69, 70, 75, 80, or any number in between) is used. In some embodiments, an oligo set that carries a random length central region of about between 6 and 66 nucleotides (i.e. 6, 7, 8, 9, 10, 15, 20, 25, 30, 35, 40, 45, 50, 55, 60, 65, 66, or any number in between) is used. In one embodiment, the oligo is flanked by two 15-30 (i.e. 15, 16, 17, 18, 19, 20, 21, 22, 23, 24, 25, 26, 27, 28, 29, or 30) nucleotide cognate regions that will be annealed to the template ssDNA. In some embodiments, the oligo set is flanked by two 20 nucleotide cognate regions that will be annealed to the template ssDNA. In some embodiments, the resulting mini library of phage that display the scFv-ligand fusion with varied connecting loop lengths is made by Kunkel mutagenesis. Other methods known in the art can be used to vary the connecting loop lengths. In some embodiments, the mini library with varied connecting loop lengths is enriched for the strongest binders by whole cell panning, and the isolated binders are sequenced.

In some embodiments, the connecting loop has known cleavable regions. For example, the connecting loop may have an enzyme cleavage site, such as, for example, a thrombin cleavage site (“GRG”).

### Antibodies

In some embodiments, the tether antibody may be a whole antibody or immunoglobulin or an antibody fragment. In some embodiments, the tether antibody is an scFv, Fab, Fab′, or IgG.

In some embodiments, the antibody contact regions include about 10-20 (i.e. 10, 11, 12, 13, 14, 15, 16, 17, 18, or 20) residues surrounding the binding site between the ligand and the functional epitope. In some embodiments, the antibody contact regions include about 13-16 (i.e. 13, 14, 15, or 16) residues surrounding the binding site between the ligand and the functional epitope.

In some embodiments, the terminal product of the method described herein is an antibody free of natural ligand, enabling the creation of an agonist and an antagonist. In some embodiments, the ligands are not limited to peptides or proteins. For example, through the use of artificial disulfide bond crosslinking, any molecule that carries a free thiol can be linked and applied to the initial screen. Since the artificial bond is phased out in the second round screening, the ligand carrying regions are also not limited to the CDRs. Thus, the ligand carrying region may be outside of the CDR regions, and may be located, for example, in the framework region.

In some embodiments, the ligand is not included in the final antibody product. Thus, the affinity of the final product does not rely on the affinity of the natural ligand. Furthermore, weaker binding ligands may perform equivalently for initial tethering and directing purposes.

An antibody of the present disclosure may be multispecific, e.g., bispecific. An antibody of the may be mammalian (e.g., human or mouse), humanized, chimeric, recombinant, synthetically produced, or naturally isolated. Exemplary antibodies of the present disclosure include, without limitation, IgG (e.g., IgG1, IgG2, IgG3, and IgG4), IgM, IgA (e.g., IgA1, IgA2, and IgAsec), IgD, IgE, Fab, Fab′, Fab′2, F(ab′)2, Fd, Fv, Feb, scFv, scFv-Fc, and SMIP binding moieties. In certain embodiments, the antibody is an scFv. The scFv may include, for example, a flexible linker allowing the scFv to orient in different directions to enable antigen binding. In various embodiments, the antibody may be a cytosol-stable scFv or intrabody that retains its structure and function in the reducing environment inside a cell (see, e.g., Fisher and DeLisa, J. Mol. Biol. 385(1): 299-311, 2009; incorporated by reference herein). In particular embodiments, the scFv is converted to an IgG or a chimeric antigen receptor according to the methods described herein.

In most mammals, including humans, whole antibodies have at least two heavy (H) chains and two light (L) chains connected by disulfide bonds. Each heavy chain consists of a heavy chain variable region (VH) and a heavy chain constant region (CH). The heavy chain constant region consists of three domains (CH1, CH2, and CH3) and a hinge region between CH1 and CH2. Each light chain consists of a light chain variable region (VL) and a light chain constant region (CL). The light chain constant region consists of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen.

Antibodies of include all known forms of antibodies and other protein scaffolds with antibody-like properties. For example, the antibody can be a monoclonal antibody, a polyclonal antibody, human antibody, a humanized antibody, a bispecific antibody, a monovalent antibody, a chimeric antibody, or a protein scaffold with antibody-like properties, such as fibronectin or ankyrin repeats. The antibody can have any of the following isotypes: IgG (e.g., IgG1, IgG2, IgG3, and IgG4), IgM, IgA (e.g., IgA1, IgA2, and IgAsec), IgD, or IgE.

An antibody fragment may include one or more segments derived from an antibody. A segment derived from an antibody may retain the ability to specifically bind to a particular antigen. An antibody fragment may be, e.g., a Fab, Fab′, Fab′2, F(ab′)2, Fd, Fv, Feb, scFv, or SMIP. An antibody fragment may be, e.g., a diabody, triabody, affibody, nanobody, aptamer, domain antibody, linear antibody, single-chain antibody, or any of a variety of multispecific antibodies that may be formed from antibody fragments.

Examples of antibody fragments include: (i) a Fab fragment: a monovalent fragment consisting of VL, VH, CL, and CH1 domains; (ii) a F(ab′)2 fragment: a bivalent fragment comprising two Fab fragments linked by a disulfide bridge at the hinge region; (iii) a Fd fragment: a fragment consisting of VH and CH1 domains; (iv) an Fv fragment: a fragment consisting of the VL and VH domains of a single arm of an antibody; (v) a dAb fragment: a fragment including VH and VL domains; (vi) a dAb fragment: a fragment that is a VH domain; (vii) a dAb fragment: a fragment that is a VL domain; (viii) an isolated complementarity determining region (CDR); and (ix) a combination of two or more isolated CDRs which may optionally be joined by one or more synthetic linkers. Furthermore, although the two domains of the Fv fragment, VL and VH, are coded for by separate genes, they can be joined, using recombinant methods, e.g., by a synthetic linker that enables them to be expressed as a single protein, of which the VL and VH regions pair to form a monovalent binding moiety (known as a single chain Fv (scFv)). Antibody fragments may be obtained using conventional techniques known to those of skill in the art, and may, in some instances, be used in the same manner as intact antibodies. Antigen-binding fragments may be produced by recombinant DNA techniques or by enzymatic or chemical cleavage of intact immunoglobulins. An antibody fragment may further include any of the antibody fragments described above with the addition of additional C-terminal amino acids, N-terminal amino acids, or amino acids separating individual fragments.

An antibody may be referred to as chimeric if it includes one or more antigen-determining regions or constant regions derived from a first species and one or more antigen-determining regions or constant regions derived from a second species. Chimeric antibodies may be constructed, e.g., by genetic engineering. A chimeric antibody may include immunoglobulin gene segments belonging to different species (e.g., from a mouse and a human).

An antibody may be a human antibody. A human antibody refers to a binding moiety having variable regions in which both the framework and CDR regions are derived from human immunoglobulin sequences. Furthermore, if the antibody contains a constant region, the constant region also is derived from a human immunoglobulin sequence. A human antibody may include amino acid residues not identified in a human immunoglobulin sequence, such as one or more sequence variations, e.g., mutations. A variation or additional amino acid may be introduced, e.g., by human manipulation. A human antibody of the present disclosure is not chimeric.

An antibody may be humanized, meaning that an antibody that includes one or more antigen-determining regions (e.g., at least one CDR) substantially derived from a non-human immunoglobulin or antibody is manipulated to include at least one immunoglobulin domain substantially derived from a human immunoglobulin or antibody. An antibody may be humanized using the conversion methods described herein, for example, by inserting antigen-recognition sequences from a non-human antibody encoded by a first vector into a human framework encoded by a second vector. For example, the first vector may include a polynucleotide encoding the non-human antibody (or a fragment thereof) and a site-specific recombination motif, while the second vector may include a polynucleotide encoding a human framework and a site-specific recombination complementary to a site-specific recombination motif on the first vector. The site-specific recombination motifs may be positioned on each vector such that a recombination event results in the insertion of one or more antigen-determining regions from the non-human antibody into the human framework, thereby forming a polynucleotide encoding a humanized antibody.

In certain embodiments, a ligand free antibody is converted from scFv to an IgG (e.g., IgG1, IgG2, IgG3, and IgG4). There are various methods in the art for converting scFv fragments to IgG. One such method of converting scFv fragments to IgG is disclosed in US patent application publication number 20160362476, the contents of which are incorporated herein by reference.

### Binding Affinity

Binding affinity for antibodies and antibody fragments can be determined through various methods known in the art. For example, binding affinity can be determined by the Scatchard analysis of Munson and Pollard, Anal. Biochem., 107:220 (1980). Another method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity, and is equal to the dissociation constant K_{d}. (See, generally, Davies et al. (1990) Annual Rev Biochem 59:439-473).

In some embodiments, the tether antibody template binding affinity to the functional epitope is greater than about a K_{d} of 1 nM. In some embodiments, the tether antibody template binding affinity to the functional epitope is about between a K_{d} of 1 and 50 nM (i.e. 1, 2, 3, 4, 5, 6, 7, 8, 9, 10, 15, 20, 25, 30, 35, 40, 45, 50, or any values in between). In some embodiments, the tether antibody template binding affinity to the functional epitope is about between a K_{d} of 1 and 15 nM (i.e. 1, 2, 3, 4, 5, 6, 7, 8, 9, 10, 11, 12, 13, 14, 15 or any values in between).

### EXAMPLES

### Example 1—Ligand-Directed Antibody Design

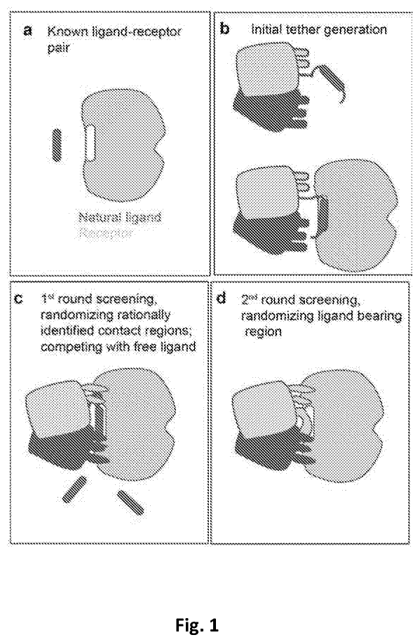

This example describes a ligand-directed antibody design strategy. An exemplary workflow for ligand-directed antibody design is depicted in FIG. 1, panels a-d. The workflow can be divided into approximately four steps. The first step is the identification of a selected ligand-receptor pair (FIG. 1, panel a). Following the identification step, a template is designed and validated (FIG. 1, panel b). This step includes the generation of the initial tether. Following initial tether generation, there is a first round screening process that occurs, wherein a first library is generated and screened, by randomizing possible contact regions of the tether to the receptor (FIG. 1, panel c). This is followed by a second library generation and screening via mutation within ligand-carrying regions (FIG. 1, panel d).

This workflow utilizes a new strategy of utilizing natural ligand affinity to generate a library of antibody variants with inherent bias toward the active site of the membrane protein. This methodology thus provides focused antibody libraries with a natural ligand encoded within or cross-linked to one of the CDRs or N-terminus. As part of this methodology, randomization of the antibody to amino acids in regions around the ligand is made while leaving the ligand carrying part unaltered to tailor binding to the active site. A second round of randomization of the ligand carrying part will then be performed to eliminate the bias of the ligand. This randomization will enable the rapid generation of functional antibodies (both agonists and antagonists) against high value targets with poor epitope exposures including GPCR and other integral membrane proteins.

### Example 2—Targeting G-Protein Coupled Receptors (GPCRs)

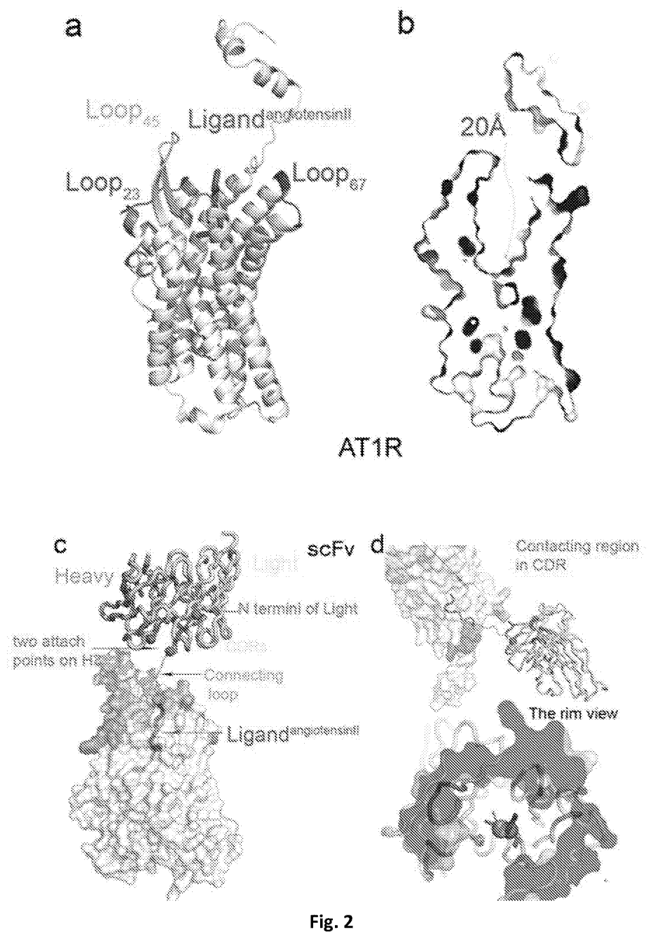

In comparison to soluble targets, or periphery membrane proteins, GPCRs possess two unique structural features that challenge the traditional antibody development. For example, GPCR's structural and functional core i.e. the 7-TM bundle (7-transmembrane bundle), only exposes very limited soluble regions (less than 20 amino acids per loop). See FIG. 2, panel a. This results in extremely low antigenicity. Often, antibodies raised against peptides from external loops or extracellular domains do not restore similar binding on the cell surface. The availability of high quality initial hits remains the primary obstacle for the early stages of development.

Another challenge is that extracellular loops of a typical GPCR are usually distant (for example, approximately 20 angstroms) from the ligand binding site which is buried deeply into the membrane and is normally inaccessible. See FIG. 2, panel b. Consequently, obtaining antibodies that not only bind but also modulate its function is highly unpredictable for the current pipeline of antibody development.

The above challenges can be addressed by the generation of a specialized ligand-directed library with a natural propensity for binding GPCR targets within the active site. This will allow for ample initial hits for downstream maturation. And the ligand directed binding will couple the affinity readout with a function throughout the screening process.

### Experimental Design

Via direct encoding (for peptidyl ligand) or disulfide linking (for non-peptidyl ligand), a functional ligand will be linked to one of the scFv CDR regions or to the C-terminus of the scFv to generate a template that weakly tethers to the membrane receptor. The binding will be evaluated using whole-cell panning pipeline, and/or other methods to determine ligand receptor binding. Using this scFv as a template, a focused screening library will be generated randomizing possible contact residues based on structure modeling. Using rolling circle amplification (RCA)-based phage library construction method will consistently generate 10¹⁰ fully recombinant libraries cost-efficiently (within 10 transformations). The initial hits will be further matured by the cost-efficient generation of a secondary library via randomizing primers covering the ligand carrying region. The final hits will be evaluated by cell-ELISA and cellular functional assays.

### Design and Validation of Initial Weakly Tethering Framework

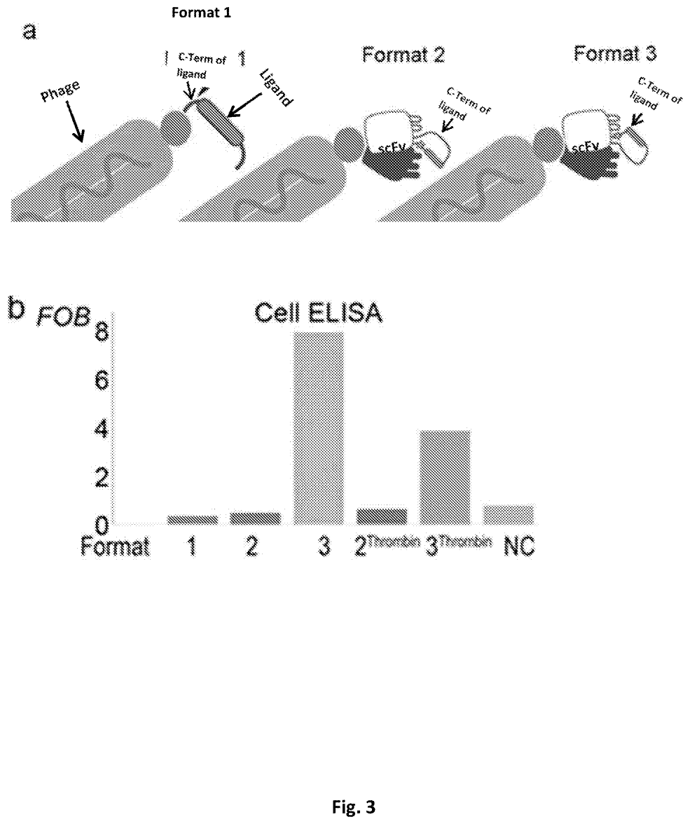

A previously developed cell surface receptor antibody (anti-Tyro3) will be used as the starting framework for generating the library and the GPCR angiotensin II receptor type 1 (AT1R) will be used as a model target. AT1R recognizes a peptidyl ligand angiotensin II (DRVYIHF) at the affinity of 10 nM, which enables us to use either genetic encoding or disulfide technology to crosslink the ligand to the antibody. Structural analysis based on the available crystal structure and scFv model (using an antibody modeling protocol established by Prof. Grey) suggests that there are potentially three different tethering points (two sites on CDR H3 and the N terminus of the light chain VL domain) and that there is a minimal length for the connecting loop. This information will be used to design several versions of the starting template and validate them using whole cell ELISA. To this end, three different phage-scFv ligand/phage-ligand formats were constructed (see FIG. 3, panels A and B). One format, which displayed binding to AT1R(+) cells and no binding to AT1R(−) cells was validated. Since this format contains engineered a thrombin cutting site at the C-terminus of the ligand peptide, thrombin treatment was performed to release one free C-terminus of the ligand peptide from the scFv. The results indicated that the digested phage-scFv displayed consistent binding activity to the target.

### Cloning and Production of Variant Versions of Initial Tethering Template

Multiple formats (e.g. six) of ligand-scFv templates will be cloned, including genetic encoding (GE clones) or free cysteine (FC clones) in all three possible tethering sites, into our phagemid vector, pIT2. In the pIT2 vector, scFv expression is induced from the lac promoter and the protein is either produced in its soluble form in the periplasm or as a fusion to the M13 phage coat protein, gpIII, if the expression strain suppresses an amber mutation between the scFv and gpIII gene (e.g. TG1). This allows for the production of phage that display multiple copies of the scFv. Phage that display multiple copies of scFv have been shown to be critical in cell-based panning and whole cell ELISA (see FIG. 5, panel c), where the increased avidity is needed for improved sensitivity. The phage particles will be pre-processed for whole cell ELISA: GE clones will be digested by thrombin to free the amino or carboxyl termini of the ligand, and FC clones will be cross-linked with the thiol containing peptidyl ligand using methods known to those of skill in the art.

Selection of Best scFv-Ligand Format by Cell ELISA

A commercial AT1R monoclonal antibody (Abcam catalog no. ab9391) will be used as the positive control and three different titrations of all six different formats will be applied to our established overexpressing AT1R cell line and parental (AT1R-negative) cell line. ELISA assays will be performed in parallel, and clones with the highest signal against AT1R positive cells and no binding to the control (AT1R-negative) cells will be chosen for subsequent processing.

### Optimization of Connecting Loop Lengths

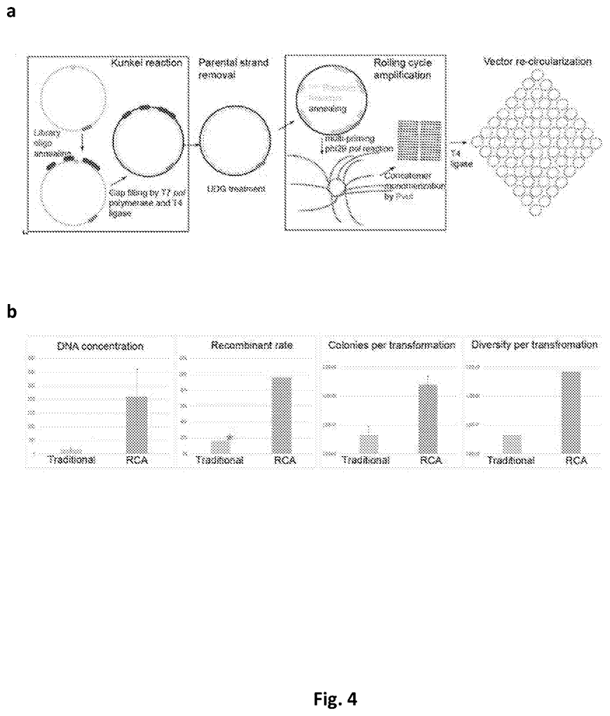

A connecting loop, that connects the ligand to the antibody, will be optimized for length. To this end, the optimal framework will be used to optimize the connecting loop lengths through screening against a mini-library that covers loop lengths that range from about 3 amino acids to about 21 amino acids. ssDNA from the template with best affinity in cell ELISA will be produced and an oligo set carrying a random length central region (6-66 nt) flanked by two 20 nt cognate regions will be annealed to the template ssDNA. A mini library of phage displaying the scFv-ligand fusion with varied connecting loop lengths will be made by Kunkel mutagenesis and screened by phage display. A schematic of Kunkel mutagenesis is shown in FIG. 4, panel a. The strongest binders will be enriched by whole cell panning and the optimal connecting loop length will be obtained by sequencing.

Optimization of ligand Binding to the Target Receptor

To have optimal binding of the ligand to the receptor, a free N-terminus or C-terminus may be required. Several ligand-linking design strategies are available, including the releasing of either terminus through thrombin treatment. See FIG. 3, panel a. Furthermore, a linker having adequate length (e.g. about 10 Å˜20 Å) will be made to ensure flexibility of the ligand.

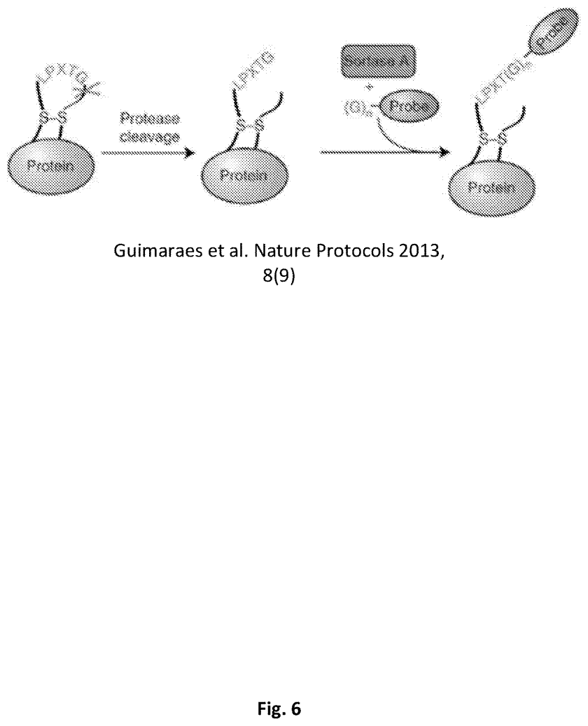

Several strategies that cross link the ligand to three potential tethering sites may be used. Two exemplary strategies include: 1) introduction of a free cysteine that is cross-linked to a NQMP-conjugated ligand on either the N- or C-terminus; 2) introduction of a sortase recognition sequence (LPXTG) at the tethering sites, which will in turn be covalently linked to an N terminal-glycine containing ligand with high efficiency when catalyzed by sortase (see FIG. 6, panels a). Using either strategy, the ligand over scFv ratios will be empirically determined to ensure maximum derivitization using anti-ligand ELISA/western blot (ab89892, Abcam).

Acquired data supports the feasibility of using whole-cell ELISA in these assays. For example, FIG. 5, panels c and d, shows anti-Tyro3 scFv that was used as the template is expressed and binds to the appropriate target-expressing cells. Additional data indicates that one of the three formats tested can bind specifically using whole cell ELISA (see FIG. 3, panel b). Furthermore, other frameworks including human/camelid nanobodies will be tested to compare with the Tyro3 scFv framework for optimal binding.

### Example 3: Library Generation and Whole Cell Panning with the Competition of Natural Ligand

The solvent exposed surface of AT1R is limited to the rim of the 7-TM helices bundle. Therefore the extra binding interface surrounding the natural ligand-receptor interface will be limited accordingly (FIG. 2, panel d). This structural feature will be utilized in generating the first round of libraries.

For the first round library, 12-15 residues will be selected that are within the contacting rim surrounding the natural ligand in our model (FIG. 2, panel d). The diversity of these residues will be referenced in sequenced human antibodies in the Kabat database.

### RCA-Based Library Construction

The first round library for the first round whole cell panning was constructed. During construction, stop codons and restriction enzyme cleavage sites were incorporated in the complementarity determining regions (CDRs) that are targeted for mutagenesis (FIG. 2, panel d), and then we used Kunkel-based site directed mutagenesis (FIG. 4) to replace these stop codons and restriction enzyme sites with tri-oligonucleotides encoding naturally distributed sets of residues at the chosen CDR positions and used Rolling Circle Amplification (RCA) to amplify the resulting library DNA. Compared with traditional libraries, RCA is far superior than traditional methods at least because both the quality and amount of DNA obtained from RCA can be greatly enhanced (See FIG. 4, panel b). Results show that a 10¹⁰ library can be made with 10 transformations.

The library of scFvs will be displayed on the surface of bacteriophage M13 as a genetic fusion to the gpIII coat protein. To ensure high avidity, a multivalent phage system was used.

### Whole Cell Based Library Screening and Validation

Water-in-oil emulsions for isolation of E. coli cells producing phage particles with desirable scFvs were used for screening. Using emulsion, the library of phage-producing E. coli cells bind to both commercial CHO and home-made human-AT1R over-expressing cells (PathHunter® CHO-K1 AGTR1 β-Arrestin Cell Line from DiscoveRx, and high-expression home-made human HEK293T lentivirus based stable AT1R cell lines) in 109 nano-droplet compartments (FIG. 5). Compared with bulk solution, micro-emulsions reduce the bias of selecting clones with higher growth rates and therefore enhance the actual diversity of initial hits. A FITC-labeled anti-M13 Ab was added to the cells, and the cells were be sorted by FACS (FIG. 5, panel b). The sorted population will be amplified and applied to the next round of emulsion screening. Three rounds of screening will be done on commercial CHO AT1R cells, and after the last round, individual clones will be validated using whole cell ELISA.

To prevent off-target binding, for each round, on-target binders will be eluted with high concentration of natural ligand in parallel of trypsin elution. In the event that cells from different species should have different off-target binders, two extra rounds of cross screening against the home made human HEK293T-AT1R expression cell lines will be performed. If needed, target-hexamerization strategy (FIG. 5, panel d) to enhance scFv-target binding avidity and/or use NGS to detect weak enrichment will be used.

### Example 3: Generation and Validation of the Second Round Library

A second round library is generated to phase out the affinity contributed by the ligand.

### Library Design and RCA Based Library Construction

Using ssDNA produced from the first round hits as template, two tightly controlled randomization strategies will be combined that introduce mutations but retain the overall sequences within the ligand carrying region, which was not altered in the first library. Libraries based on two different strategies are made and mixed as the second round library. The two different randomization strategies are: 1) a 2-5% mutation rate is introduced to the ligand and its flanking region (˜150 nt in total) using an error-prone PCR protocol in an affinity maturation pipeline; and 2) Segmental randomization is performed, wherein an NNK randomization scanning window of 9 nt (or 3 aa) will be applied on the ligand and −4aa and +4aa flanking sequences (˜45 nt in total, 5 randomizing oligoes will be used). The obtained DNA will be used to generate a second library using the same RCA-based protocol described above, and shown in FIG. 4, panel a. Phage particles will be produced and applied to whole cell screening for multiple rounds using competition with the natural peptide ligand.

### Antibody Characterization

440 or more individual clones isolated from the screen will be analyzed by whole cell phage ELISA against the AT1R (+) and AT1R (−) cells. Clones with an ELISA signal >2-fold over background will be expressed in E. coli and purified by metal affinity chromatography.

### Functional Validation

The soluble scFvs will be further validated by the beta-arrestin recruitment assay using the PathHunter® CHO-K1 AGTR1 β-Arrestin Cell Line from DiscoveRx. Activation of AT1R will be quantitated by enzymatic activity due to β gal complementation. Agonist scFvs will be detected by the activation signal of β arrestin recruitment when the scFv is applied to the cells. Antagonist scFvs will be detected by pre-incubation of the cells with the scFvs, followed by addition of angiotensin II. The activation signal in the presence of the scFv will be compared with the scFv-free angiotensin II activation signal. The validated scFvs will be converted to full immunoglobulins (IgG) by cloning the variable heavy and light chain genes into vectors and expressing them by transient transfection of HEK-293 or CHO cells.

### AXM Affinity Maturation

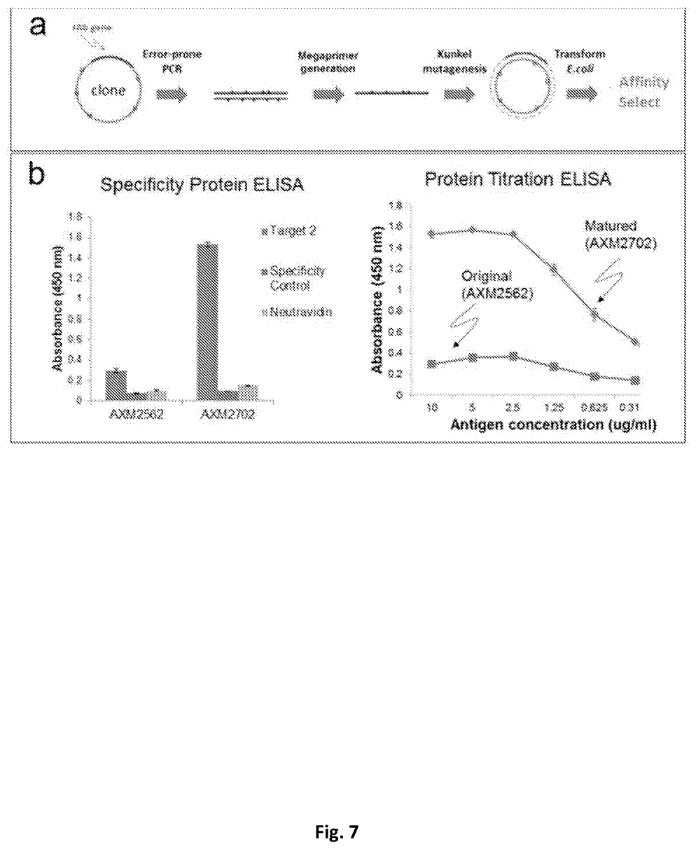

In the event that the scFv binding properties are weak (e.g. micromolar affinity or higher), the lengths of the linkers that connect the ligand to the scFv will be varied to achieve optimal, increased binding. Additionally, AXM affinity maturation mutagenesis will be performed (see FIG. 7, panels a and b). scFv antibodies that show weak recognition of the target can be affinity matured in parallel in as little as about 4 weeks. Candidate scFvs can be converted to IgG prior to this validation if required.

In the AXM affinity maturation mutagenesis process (FIG. 7, panel a), the coding region for the recombinant antibody (rAb) is amplified under error-prone PCR conditions, using a reverse primer containing exonuclease-resistant linkages on its 5′ end. The resulting double-stranded DNA is treated with 5′→3′ exonuclease to selectively degrade the unmodified-primer strand of the dsDNA molecule. The resulting single-stranded DNA is then annealed to a uracilated, circular, single-stranded “master” phagemid DNA template containing 6 Sac II sites (one in each of the 6 CDRs) and used to prime in vitro synthesis by DNA polymerase. The ligated, heteroduplex product is then transformed into E. coli TG1 cells encoding the SacII isoschizomer restriction endonuclease Eco29kI. The uracilated-, SacII-parental-strand is cleaved in vivo by Eco29kI and uracil N-glycosylase, favoring survival of the newly synthesized, recombinant strand. By using a common set of primers in Kunkel mutagenesis the need to synthesize expensive custom mutagenic primers is avoided. Transformation of the circular product from Kunkel mutagenesis into E. coli is highly efficient. Subcloning and ligation into a vector as used in error-prone PCR mutagenesis is highly inefficient. There is approximately a 100-1000× greater efficiency in generating large error-prone libraries by eliminating the subcloning step of error-prone PCR.

### Example 4: Generation and Validation of NTSR1 and NTSR2 Ligand Libraries

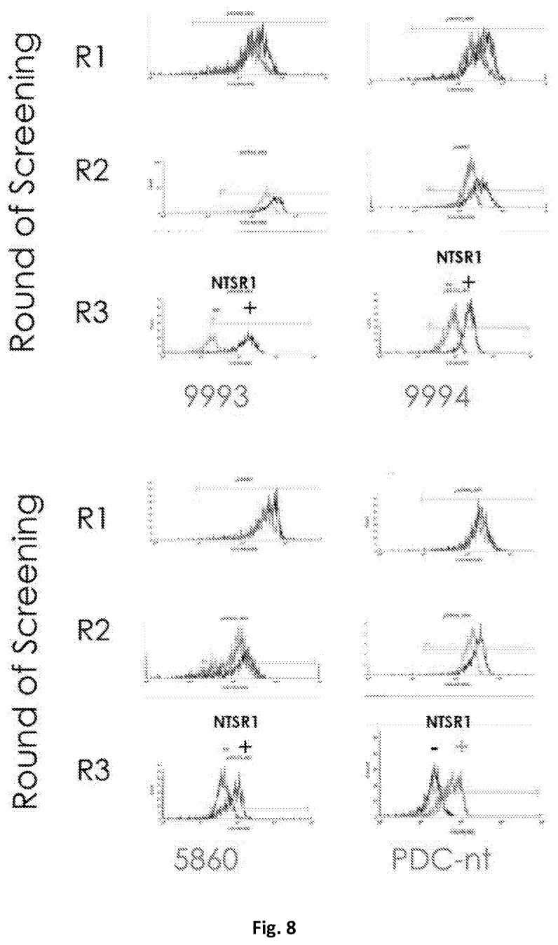

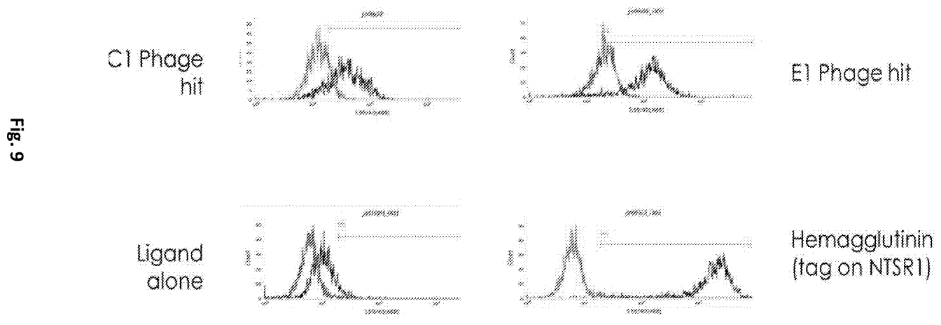

Using the above-described methods, ligand libraries were developed for the Neurotensin Receptor Type 1 (NTSR1) and Neurotensin Receptor Type II (NTSR2). Analyses of the ligand libraries were performed. FACS analysis of the NTSR1 libraries indicated that increased rounds of selection resulted in increased separation of binding antibodies (FIG. 8). For these experiments, NTSR1 ligand library was screened against control and NTSR1+ cells. The data presented in FIG. 8 shows the improvement of panning with rounds using polyclonal phage FACS FITC assay for multiple panning on NTSR1. FACS antiM13 FITC signal (1:500 dilution of anti M13 FITC conjugated antibody (catalog no. ab24229 Abcam)) for eluted polyclonal phage. After each bio-panning rounds, phage precipitated from 50 ml overnight E. coli cultures transduced with multivalent helper phage (M13 K07ΔpIII catalog no. PRHYPE from PROGEN) with MOI 20:1) were incubated with 0.5M target cells or parental cells for 1 hour at room temperature, and washed by phosphate buffered saline (PBS) with 0.01% tween, incubated with anti M13 FITC for 1 h at room temperature and washed with PBS. Cells were resuspended for FACS analysis. 2000 events were collected on a BD Jazz Flow cytometer, debris and duplet were filtered via appropriate gating using forward scatter and side-scattering limits. Histograms (y axis representing counts of cells and x axis representing FITC fluorescence signal) were generated” as colored curves represent parental HEK293T cells as a negative control, and black curves represent target expression HEK293T cells. Panning of 3 rounds (R1, R2 and R3) via 4 libraries (9993, 9994,5860 and PDC-nt) showed an increasing contrast of signal between the parental cells and target cells, indicated by the increasing distances between positions of positive and negative peaks.

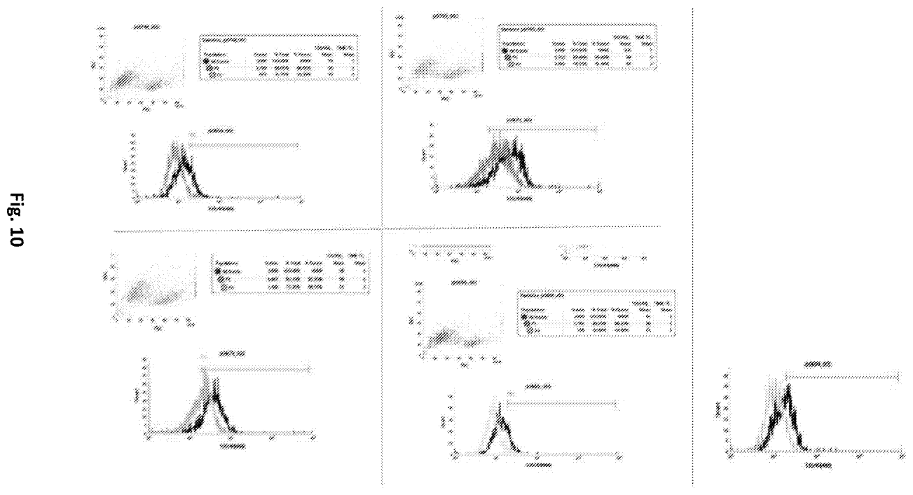

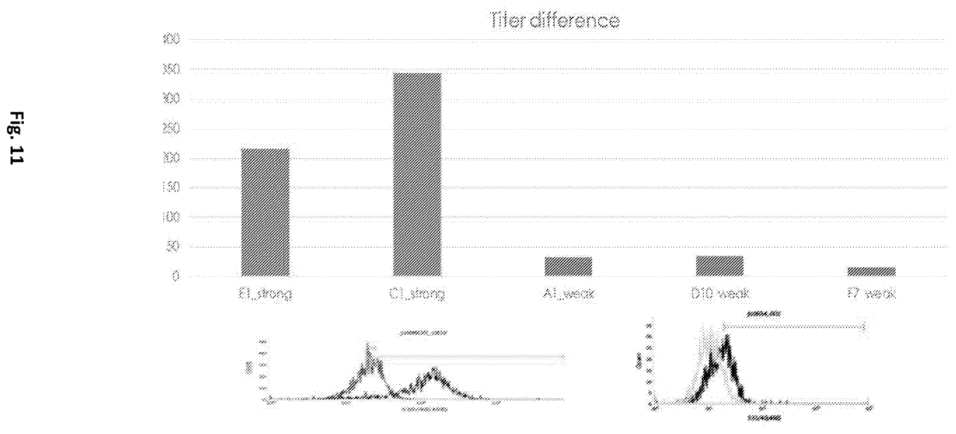

Data obtained by FACS analysis of exemplary, strong, anti-NTSR1 phage hits is shown in FIG. 9.

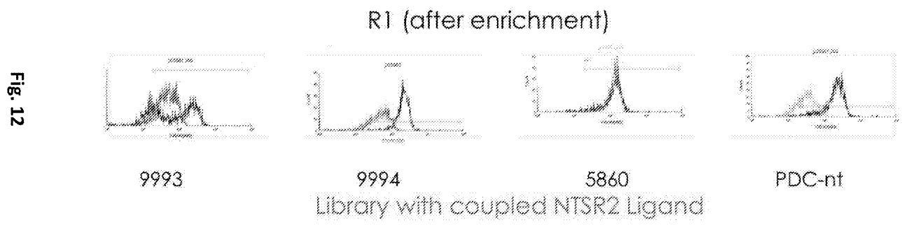

Analyses of the NTSR1 library indicated that weak hits are more abundant than strong hits. Representative FACS graphs of weak binders are shown in FIG. 10. Further analyses of phage titers between weak and strong hits revealed a difference in phage titer between the two groups. Phage titer difference between strong and weak hits are about 10-30 fold (FIG. 11). For subsequent assays, fewer cells can be used to prevent competition of 106 NTSR1 receptors per cell.

Validation of NTSR2 ligand libraries was also performed. FACS data obtained from these experiments are presented in FIG. 12. The data indicate good separation (e.g. high specificity) of ligand-containing libraries.

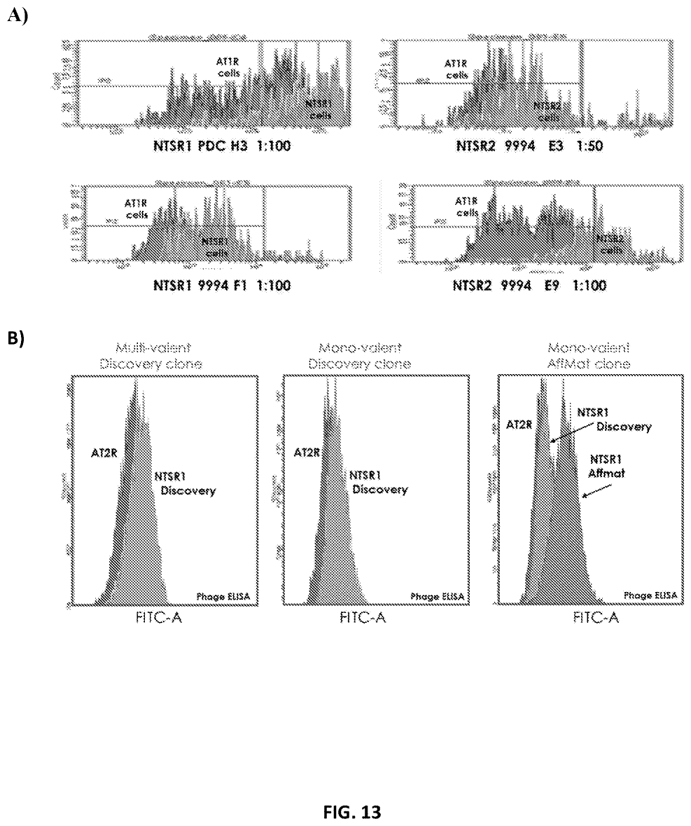

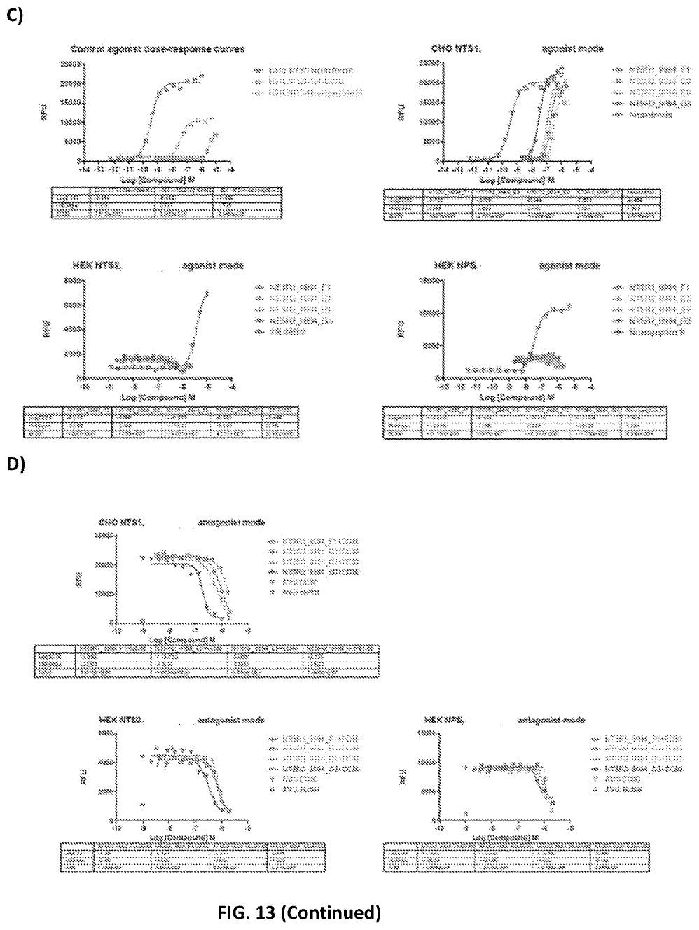

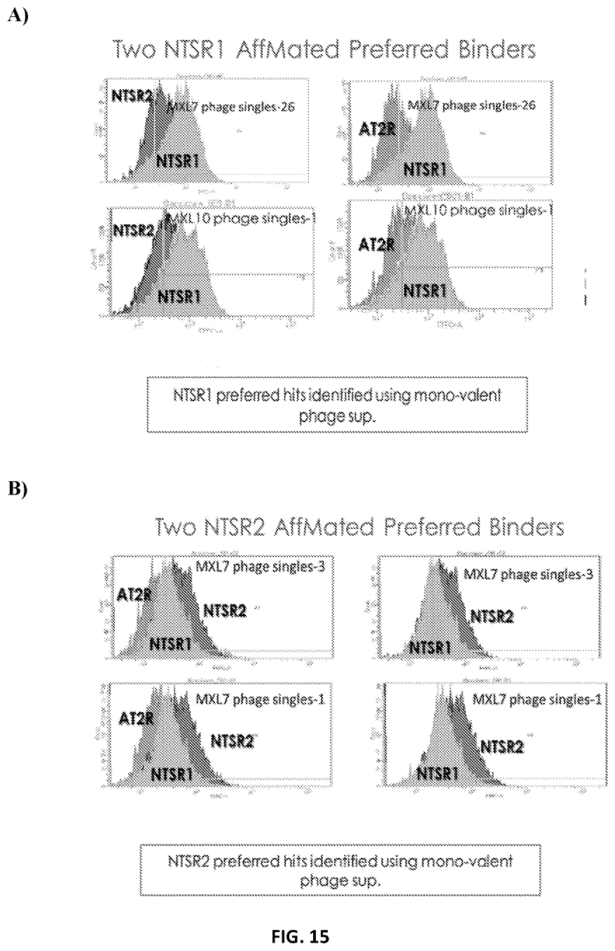

Several rounds of validations of the NTSR1 and NTSR2 isolated antibodies were performed, including flow cytometry analysis and functional activity measures (FIG. 13A-D). The data from these assays show strong, highly specificity of NTSR1 or NTSR2 binders in flow cytometry assays (FIG. 13A). The effects of affinity maturation were also tested to assess whether affinity maturation resulted in improved binding. The results show that affinity maturation has a marked effect on the binding specificity and strength of the isolated NTSR1 binders (FIG. 13B and FIGS. 15A and 15B).

The functionality of the NTSR1 and NTSR2 isolated binders were assessed in functional assays. For these assays, cells were incubated with the isolated binders in NPS calcium assays under either an agonist mode or an antagonist mode. The data show that NTSR2 antagonists agonize NTSR1 cells, and that NTSR1 agonist antagonize NTSR2 cells (FIGS. 13C and D).

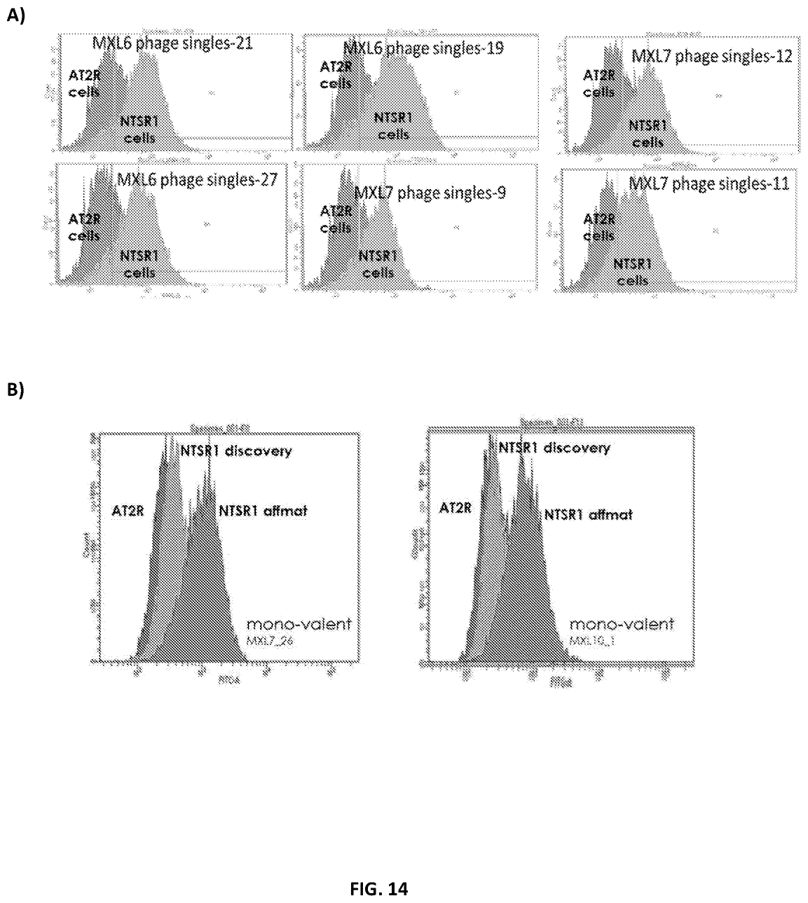

Binding assays and FACS analysis with isolated NTSR1 binders showed that affinity matured NTSR1 binders bound with high affinity as monovalent phage on NTSR1 cells. The data also showed that affinity matured phage bind tighter than non-affinity matured phage. (FIG. 14, panels A and B). For these experiments, monovalent phage supernatant (200 uL with 1% BSA) that was transduced with KM13 and incubated with irrelevant GPCR cells AT2R and relevant GPCR NTSR1. Monovalent discovery clone phage shifts slightly away from phage on irrelevant cells but after affinity maturation (“affmat”) procedure has increased the shift denoting better binding of the phage to the relevant NTSR1 expressing cells. FACS analysis was performed and validated by standard methods.

### Example 5: Generation and Functional Validation of CXCR4 Antibodies

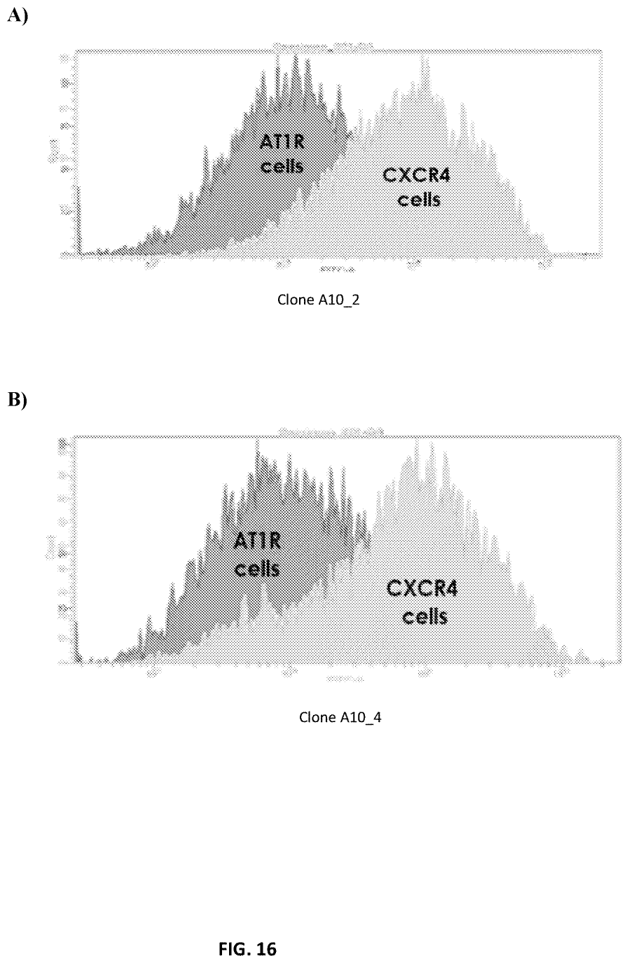

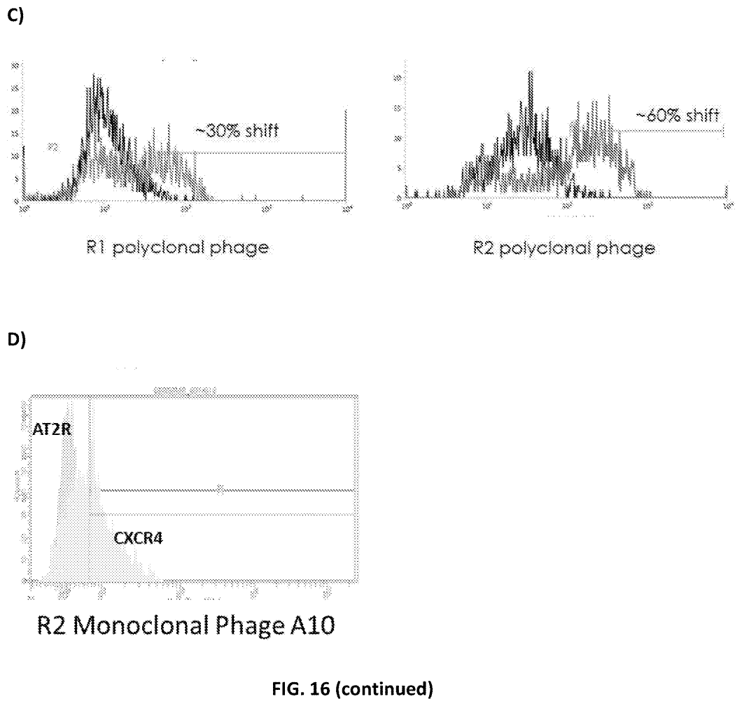

Binders were also developed against the GPCR, CXCR4 in accordance to the methods provided herein. CXCR4 polyclonal phage were isolated and binding was confirmed by FACS analysis (FIG. 16, panel C). The data from the FACS analysis showed that the phage bound after bulk panning and one round of whole cell panning. The amount of phage binding increased after a second round of panning (FIG. 16, panel C). For the panning procedures, both bulk panning and negative panning was performed, followed by sorting/positive selection, and FACS analysis as a quality control measure.

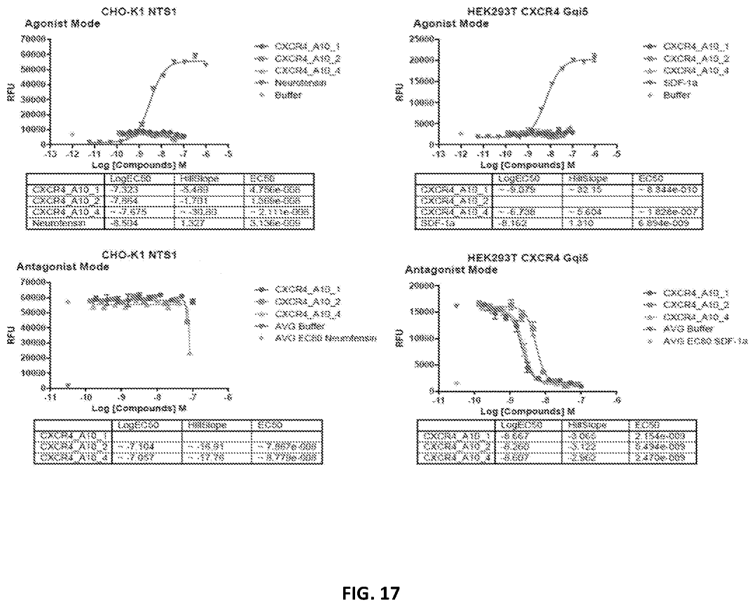

CXCR4 binders were identified, which were converted to IgGs, purified and assayed for binding and functional testing (FIG. 16A-16C and FIG. 17). The monoclonal phage sequences were cloned into IgGs for FACS analysis. For these assays, IgG were incubated with AT2R cells that expressed an irrelevant GPCR and a AT2R cells that expressed CXCR (i.e. GPCR of interest). The cells were processed for flow cytometry analysis using standard methods. Clones CXCR4 A10_2 and CXCR4 A10_4 showed good binding as IgGs. The data indicated high specificity and separation of the signals in both of the isolated CXCR4 binders (FIGS. 16A and 16B).

Functional assays indicated that CXCR4 binders are strong antagonists. The functional assay incorporated the use of both control cells (cells that over-expressed an irrelevant peptide) and cells that expressed CXCR4. CXCR4 calcium assay agonist and antagonist modes were performed in accordance with standard methods. The data obtained from these assays confirm that the isolated binders are functional (FIG. 17).

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method of generating antibodies against a target protein, comprising:
(a) providing a tether antibody template comprising an antigen binding region and a ligand that binds to an epitope of a target protein;
(b) generating a first library by randomizing one or more contact regions of the antigen binding region adjacent to a binding site between the ligand and the epitope;
(c) screening the first library to identify one or more antibodies with improved binding affinity to the epitope as compared to the ligand;
(d) generating a second library by randomizing a ligand carrying region of the one or more antibodies identified in step (c);
(e) screening the second library to identify one or more antibodies that bind to the target protein with the same or improved affinity as compared to the ligand.

2. The method of claim 1, wherein the epitope is a functional epitope.

3. The method of claim 1, wherein the generated antibody is an agonist or antagonist.

4. The method of claim 1, wherein the generated antibody is not an agonist or antagonist.

5. The method of claim 1, wherein the target protein is a membrane protein.

6. The method of claim 5, wherein the membrane protein is a transmembrane receptor, enzyme or structural protein.

7. The method of claim 6, wherein the transmembrane receptor is a G-protein coupled receptor (GPCR), ion channel-coupled receptor, viral receptor, or enzyme-linked protein receptor.

8. The method of claim 6, wherein the enzyme-linked protein receptor is a receptor tyrosine kinase.

9. The method of any one of claims 2-8, wherein the functional epitope is an active site.

10. The method of claim 9, wherein the active site is a ligand binding site.

11. The method of claim 9, wherein the active site is a catalytic site.

12. The method of any one of the preceding claims, wherein the antigen binding region of the tether antibody template is fused to the ligand via a peptide bond.

13. The method of any one of claims 1-12, wherein the antigen binding region of the tether antibody template is conjugated to the ligand via a covalent bond.

14. The method of claim 13, wherein the covalent bond is a disulfide bond.

15. The method of claim 13, wherein the tether antibody is conjugated by a sortase or a transglutamase.

16. The method of any one of the preceding claims, wherein the antigen binding region of the tether antibody is an antibody fragment.

17. The method of any one of the preceding claims, wherein the antigen binding region of the tether antibody template is a scFv, Fab, Fab′, or IgG.

18. The method of any one of the preceding claims, wherein the antigen binding region of the tether antibody template is a scFv.

19. The method of any one of the preceding claims, wherein the ligand is a peptide.

20. The method of any one of claims 1-18, wherein the ligand is a small molecule compound.

21. The method of any one of the preceding claims, wherein the ligand is fused or conjugated to a CDR of the antigen binding region.

22. The method of any one of claims 1-20, wherein the ligand is fused or conjugated to the N-terminus or C-terminus of a light chain variable region.

23. The method of any one of claims 1-20, wherein the antigen binding region is a scFv and the ligand is fused or conjugated to the C-terminus of scFv.

24. The method of any one of the preceding claims, wherein the ligand is fused or conjugated via its N-terminus or C-terminus to the antigen binding region.

25. The method of any one of the preceding claims, wherein there is a connecting loop between the antigen binding region and the ligand.

26. The method of claim 25, wherein the connecting loop is a peptide.

27. The method of claim 26, wherein the peptide comprises 3-50 amino acids.

28. The method of claim 26, wherein the peptide comprises 3-21 amino acids.

29. The method of claim 25, wherein the connecting loop is a protein.

30. The method of any one of claims 23-29, wherein the method further comprises a step of optimizing the connecting loop.

31. The method of claim 30, wherein the step of optimizing the connecting loop comprises screening a mini-library comprising a plurality of peptides with various lengths.

32. The method of any one of claims 25-31, wherein the connecting loop comprises an enzyme cleavage site.

33. The method of claim 32, wherein the enzyme cleavage site is a thrombin cleavage site.

34. The method of any one of the preceding claims, wherein prior to step (a), the method further comprises steps of
designing a plurality of candidate tether antibody templates; and
selecting the tether antibody template with desired binding affinity to the functional epitope.

35. The method of claim 34, wherein the designing step comprises structural analysis of the antigen binding region and/or the ligand.

36. The method of claim 34, wherein the plurality of candidate tether antibody templates are presented by phage display.

37. The method of claim 37, wherein the plurality of candidate tether antibody templates are expressed as a soluble protein in the periplasm.

38. The method of claim 37, wherein the plurality of candidate tether antibody templates are expressed as a fusion to the M13 phage coat protein gpIII.

39. The method of any one of claims 33-38, wherein the selecting step comprises whole cell panning.

40. The method of any one of claims 33-39, wherein the selecting step comprises whole cell ELISA.

41. The method of any one of claims 34-40, wherein the desired binding affinity of the selected tether antibody template to the functional epitope has a k_{d} greater than 10 nM

42. The method of any one of the preceding claims, wherein the one or more contact regions comprise 13-16 residues surrounding the binding site between the ligand and the functional epitope.

43. The method of any one of the preceding claims, wherein the one or more contact regions are randomized by
incorporating one or more stop codons and/or restriction enzyme cleavage sites,
replacing the one or more stop codons and/or restriction enzyme sites by site directed mutagenesis resulting in a DNA template, and
amplify the resulting DNA template by rolling circle amplification (RCA), thereby generating the first library.

44. The method of claim 43, wherein the RCA is error-prone RCA.

45. The method of any one of the preceding claims, wherein the one or more contact regions are randomized without altering the ligand carrying region of the tether antibody template.

46. The method of any one of the preceding claims, wherein the first library is a phage display library.

47. The method of any one of the preceding claims, wherein the first library has a diversity of at least 10⁸, 10⁹, 10¹⁰, 10¹¹, or 10¹².

48. The method of any one of the preceding claims, wherein the step of screening the first library comprises whole cell panning.

49. The method of claim 48, wherein the whole cell panning is emulsion based.

50. The method of any one of claim 48 or 49, wherein the one or more antibodies with improved binding affinity to the functional epitope are selected by competition assay using free ligands.

51. The method of any one of the preceding claims, wherein the second library is a phage display library.

52. The method of any one of the preceding claims, wherein the second library is generated by RCA.

53. The method of claim 52, wherein the RCA is error-prone RCA.

54. The method of claim 53, wherein the error-prone RCA has 1-10% mutation rate.

55. The method of any one of the preceding claims, wherein the screening step of the second library comprises whole cell panning.

56. The method of any one of the preceding claims, wherein the method further comprises a step of validating the one or more ligand free antibodies identified in step (e).

57. The method of claim 56, wherein the one or more ligand free antibodies are validated by a functional assay.

58. The method of claim 56 or 57, wherein the step of validating the one or more ligand free antibodies identified in step (e) comprises converting scFv to IgG.

59. The method of any one of the preceding claims, wherein the method further comprises determining if the one or more ligand free antibodies are antagonistic or agonistic antibodies.

60. A functional antibody against a target protein of interest generated according to a method of any one of the preceding claims.

61. A first library generated according to a method of any one of the preceding claims.

62. A second library generated according to a method of any one of the preceding claims.

63. A library comprising a plurality of tether antibodies comprising an antigen binding region and a ligand that binds to a target protein, wherein the plurality of tether antibodies are derived from a tether antibody template and comprise randomized one or more contact regions adjacent to a binding site of the ligand and an epitope of the target protein.

64. The library of claim 63, wherein the epitope is a functional epitope.

65. The library of claim 63, wherein the plurality of tether antibodies comprise an unaltered ligand carrying region.

66. The library of claim 63 or 65, wherein the antigen binding region is fused to the ligand via a peptide bond.

67. The library of claim 63 or 65, wherein the antigen binding region is conjugated to the ligand via a covalent bond.

68. The library of claim 67, wherein the covalent bond is a disulfide bond.

69. The library of any one of claims 63-68, wherein the antigen binding region is an antibody fragment.

70. The library of any one of claims 63-68, wherein the antigen binding region is a scFv, Fab, Fab′, or IgG.

71. The library of claim 70, wherein the antigen binding region is a scFv.

72. The library of any one of claims 63-71, wherein the ligand is a peptide.

73. The library of any one of claims 63-71, wherein the ligand is a small molecule compound.

74. The library of any one of claims 63-71, wherein the ligand is a polymer, DNA, RNA or sugar.

75. The library of any one of claims 63-71, wherein the ligand is fused or conjugated to a CDR of the antigen binding region.

76. The library of any one of claims 63-71, wherein the ligand is fused or conjugated to the N-terminus or C-terminus of a light chain variable region.

77. The library of any one of claims 63-71, wherein the antigen binding region is a scFv and the ligand is fused or conjugated to the C-terminus of scFv.

78. The library of any one of claims 63-77, wherein the ligand is fused or conjugated via its N-terminus or C-terminus to the antigen binding region.

79. The library of any one of claims 63-77, wherein there is a connecting loop between the antigen binding region and the ligand.

80. The library of claim 79, wherein the connecting loop is a peptide.

81. The library of claim 80, wherein the peptide comprises 3-50 amino acids.

82. The library of claim 81, wherein the peptide comprises 3-21 amino acids.

83. The library of any one of claims 79-82, wherein the connecting loop comprises an enzyme cleavage site.

84. The library of claim 83, wherein the enzyme cleavage site is a thrombin cleavage site.

85. The library of any one of claims 63-84, wherein the library is a phage display library.

86. The library of claim 75, wherein the plurality of tether antibodies are expressed as a soluble protein in the periplasm.

87. The library of claim 85, wherein the plurality of tether antibodies are expressed as a fusion to the M13 phage coat protein gpIII.

88. The library of any one of claims 63-87, wherein the library has a diversity of at least 10⁸, 10⁹, 10¹⁰, 10¹¹, or 10¹².

89. A library comprising a plurality of candidate antibodies for binding to a target protein, wherein the plurality of the candidate antibodies are derived from a parent antibody comprising one or more contact regions adjacent to a binding site between a ligand and an epitope of the target protein and a ligand carrying region that contacts the epitope and competes with the ligand, wherein the plurality of candidate antibodies comprise randomized ligand carrying region.

90. The library of claim 89, wherein the epitope is a functional epitope.

91. The library of claim 89, wherein the plurality of candidate antibodies comprise substantially identical one or more contact regions.

92. The library of claim 89 or 91, wherein the library is a phage display library.

93. The library of claim 92, wherein the plurality of candidate antibodies are expressed as a soluble protein in the periplasm.

94. The library of claim 92, wherein the plurality of tether antibodies are expressed as a fusion to the M13 phage coat protein gpIII.

95. The library of any one of claims 89-94, wherein the library has a diversity of at least 10⁸, 10⁹, 10¹⁰, 10¹¹, or 10¹².

96. A method of generating binders against a target protein, comprising:
(a) providing a tether antibody template comprising an antigen binding region and a ligand that binds to an epitope of a target protein;
(b) generating a first library by randomizing one or more contact regions of the antigen binding region adjacent to a binding site between the ligand and the epitope;
(c) screening the first library to identify one or more binders with improved binding affinity to the epitope as compared to the ligand;
(d) generating a second library by randomizing a ligand carrying region of the one or more binders identified in step (c);
(e) screening the second library to identify one or more binders that bind to the target protein with the same or improved affinity as compared to the ligand.

97. The method of claim 96, wherein the ligand is a peptidomimetic or aptamer.
